(19) United States
(12) Patent Application Publication
(10) Pub. No.: US 2010/0261229 A1
(43) Pub. Date: Oct. 14, 2010

(54) SYSTEM AND METHOD FOR PREPARING AND USING BULK EMULSION

(75) Inventors: Aldrich N. K. LAU, Palo Alto, CA (US); Christine LAMBERT, San Mateo, CA (US); Achim KARGER, Foster City, CA (US); Madison JORDAN, Foster City, CA (US)

Correspondence Address: KILYK & BOWERSOX, P.L.L.C., 3925 CHAIN BRIDGE ROAD, SUITE D401, FAIRFAX, VA, 22030, US

(73) Assignee: APPLIED BIOSYSTEMS, LLC, Carlsbad, CA (US)

(21) Appl. No.: 12/756,547

(22) Filed: Apr. 8, 2010

Related U.S. Application Data
(60) Provisional application No. 61/307,428, filed on Feb. 23, 2010.
(60) Provisional application No. 61/167,781, filed on Apr. 8, 2009.
(60) Provisional application No. 61/167,766, filed on Apr. 8, 2009.

(51) Int. Cl. C12P 19/34; C12M 1/00

(52) U.S. Cl. 435/91.2; 435/289.1

(57) ABSTRACT

An emulsion generation apparatus and method for forming an emulsion are provided wherein a customized impeller design is adapted to form an emulsion with a desired droplet size that defines a desired volume. The emulsion generation apparatus provides improved uniformity in emulsion preparation and may be used to create large or small volume emulsions rapidly and reproducibly. A system and method are also provided for large volume sample amplification adaptable for use with conventional PCR-based reactions as well as emulsion-based PCR reactions and other reactions. For applications involving emulsion-based PCR amplification, the system and method provide improved uniformity in emulsion amplification and can be used to amplify large or small volume emulsions rapidly and reproducibly.

### CROSS-REFERENCE TO RELATED APPLICATIONS

The present application claims the benefit of the earlier filing date of U.S. Provisional Patent Applications Nos. 61/307,428, filed Feb. 23, 2010, 61/167,781, filed Apr. 8, 2009, and 61/167,766, filed Apr. 8, 2009, each of which is incorporated herein in its entirety by reference.

### FIELD

The present teachings relate to devices, systems, and methods for preparing and reacting within emulsions, including emulsions useful in biological reaction processes, for example, useful in a polymerase chain reaction (PCR).

### INTRODUCTION

A number of biological sample analysis methods rely on sample preparation steps as a precursor to carrying out the analysis methods. For example, a precursor to performing many biological sequencing techniques (e.g., sequencing of nucleic acid) includes amplification of nucleic acid templates in order to obtain a large number of copies (e.g., millions of copies) of the same template.

Polymerase chain reaction is a well understood technique for amplifying nucleic acids which is routinely used to generate sufficiently large DNA populations suitable for downstream analysis. Recently, PCR-based methods have been adapted to amplifying samples contained within emulsions for sequencing applications. In such amplification methods a plurality of biological samples (e.g. nucleic acid samples) may be individually encapsulated in microcapsules of an emulsion and PCR amplification conducted on each of the plurality of encapsulated nucleic acid samples simultaneously. Such microcapsules are often referred to as “microreactors” because the amplification reaction occurs within the microcapsule.

In some cases, the microcapsule can include a template bead, also referred to as a P1 bead or a primer 1 bead and the amplification process may be referred to as bead-based emulsion amplification, for example, as described in US 2008/0003571 Al to McKernan et al., which is incorporated herein in its entirety by reference. In such a technique, beads along with DNA templates are suspended in an aqueous reaction mixture and then encapsulated in an inverse (water-in-oil) emulsion. The template DNA may be either bound to the bead prior to emulsification or may be included in solution in the amplification reaction mixture. For further details regarding techniques for bead emulsion amplification, reference is made to PCT publication WO 2005/073410 A2, entitled “NUCLEIC ACID AMPLIFICATION WITH CONTINUOUS FLOW EMULSION,” which published internationally on Aug. 11, 2005, and is incorporated by reference in its entirety herein.

According to various methodologies, performing bead-based emulsion amplification relies on the formation of an emulsion which encapsulates a template DNA strand, a bead upon which DNA strands amplified from the template DNA strand are retained and a reagent mixture for supporting the amplification reaction. As noted above, the emulsion typically comprises an inverse (water-in-oil) emulsion with the aqueous phase (e.g., dispersed phase) including the reagent mixture and the beads, and the continuous phase including oil or other non-aqueous liquid partially or completely immiscible in water.

Various emulsion preparation techniques have been used. For example, WO 2005/073410 A2, incorporated by reference herein, teaches a cross-flow emulsification system in which emulsion oil is pumped into one of a plurality of tees having a tapered area that is in flow communication with a syringe configured to inject a plurality of microreactors into the emulsion oil to form the emulsion. This system may generate droplets of 80 to 120 μm with the dispense channel diameter of 120 μm. Therefore, the droplet size is generally comparable to the dispense channel size. Using such a system one may encounter difficulties in employing the described cross-flow system to generate smaller droplets for example below 10 μm (including in the range of 4 to 9 μm) in diameter. Considerations in this regard is that manufacture of tees with channels smaller than 10 μm may be expensive and the emulsification may take an long time due to a generally low flow rate that can be achieved through the such dispense channel. In addition, the process may require application of high pressure to push the PCR mixture with the beads through the narrow opening, and may in turn limit the choice of materials capable to withstand the applied pressure. Such systems may also be prone to clogging and beads sedimentation.

An emulsification system based on agitation of the continuous phase may address some of the aforementioned issues and allow for various methods of the dispersed phase addition. One technique (Dressman et al, PNAS, Jul. 22, 2003, vol. 100, no. 15, 8817-8822) describes a method for emulsion preparation using a magnetic stirrer and a magnet bar agitating the continuous oil phase while aqueous phase (PCR mixture with beads) is being added dropwise to it using a manual pipette. A drawback of this system is that it may be difficult to obtain a desired droplet size or to control the range of droplet sizes present in the emulsion and can result in poor uniformity and reproducibility of the emulsion. Finally, in this system, magnetic beads may become oriented in the strong magnetic field of the stirrer, thus resulting in a non-random bead distribution in the emulsion.

In another method of emulsification, a more complex approach was taken (Diehl et al., PNAS, Nov. 8, 2005, vol. 102, no. 45, 16368-16373). Initially, both aqueous and oil phases were mixed together (no dispensing) and briefly vortexed followed by quick emulsification using an overhead homogenizer. This process involves multiple steps and at least two transfers of emulsion from one vessel into another, which can lead to sample losses. Furthermore, there is also a concern that existing disposable emulsion generators may not be effective in making uniform emulsions with the optimum droplet size on the scale larger than 1 mL. In addition, the drop size and size distribution of emulsions prepared in such manner vary from batch to batch. Combining numerous small batches into one large batch results in broad size distribution with multiple populations of drop sizes. Consequently, PCR performance of such combined emulsions is non-reproducible.

Conventional methods typically have low throughput in emulsion generation. In general, microchannels and track-etched membranes may take hours or days to generate 1×10¹⁰ or more aqueous droplets.

Thus, conventional emulsion preparation techniques may be relatively time-consuming. In addition, such conventional techniques are relatively user-intensive, requiring the user to perform iterative pipetting, or other dispersion phase adding steps and vortexing steps and/or to hold the test tube in position as it is being vortexed. Further, the iterative process of the dispersion phase adding steps and the vortexing steps may be labor intensive under conventional methods since the user typically removes the test tube from the vortex mixer during the dispersion phase adding step. Using magnetic forces to agitate the emulsion may be detrimental to the emulsion quality. Overhead homogenizers require multiple transfers of the emulsion and may not be suitable for making emulsions on large scales, for example, larger than 10 mL.

A significant consideration for a sequencing workflow using emulsions relates to the amplification of DNA within individual microreactors once the emulsion has been formed. A typical emulsion preparation for a sequencing reaction may have a volume of approximately 1 ml or less. Such relatively small volumes may be retained in a standard microtube (for example with a volumetric capacity of approximately 1 ml, 1.7 ml, or 2 ml). These microtubes are of a size and dimensionality to reside within the thermal block of commercially available thermocyclers such as the Applied Biosystems 9700 thermocycler. Amplification of the constituents present in the emulsion by polymerase chain reaction may then be conducted according to known methods.

Various problems arise, however, where the desired emulsion volume exceeds the capacity of the microtubes used with conventional thermocyclers. For example, for a larger volume emulsion preparation it may be necessary to prepare separate emulsions or distribute aliquots in separate microtubes to be thermal cycled independently. Consequently, additional effort and care must be taken when preparing and reacting large volume emulsions increasing the amount of time and labor involved to achieve the desired amplification. Furthermore, each portion of the subdivided emulsion may be subject to increased variability arising from the local reaction characteristics which may differ from one microtube to the next (for example due to thermal variability within the block of the thermal cycler).

It will be appreciated that the step of amplification of the emulsion through PCR (ePCR) is an important step in many next generation sequencing workflows. Oftentimes, a sample to be amplified and sequenced is relatively precious and loss or inefficient sample amplification is not acceptable. In those emulsion preparations where a relatively large emulsion volume is to be amplified the manner and apparatus in which the ePCR is conducted becomes significant.

Another aspect of ePCR reactions occurring in relatively large volumes relates to the heat transfer characteristics of the reaction which is different from that of conventional (aqueous phase only) PCR reactions where the reagents for the conventional PCR reactions have fluidic properties similar to that of water alone. Large volume ePCR therefore should take into consideration the multiphase composition and characteristics of the fluidic constituents (e.g. aqueous and non-aqueous phases) which may possess different fluidic properties affecting the manner in which the temperature ramping of the reaction is conducted. For the reasons discussed above large volume emulsion amplification may benefit from a different engineering solution from that of the traditional smaller volume PCR-based reactions.

It is therefore desirable to provide a more convenient emulsion preparation technique, for example, one that reduces the activity required by a user during formation of the emulsion or may be suitable to automate. It also may be desirable to provide an emulsion preparation technique that facilitates increasing the throughput of biological sample analysis processes by increasing the efficiency of sample preparation including increasing the capacity of emulsion preparation for volumes over 10 ml.

Moreover, it may be desirable to provide an emulsion preparation technique that yields emulsions with increased consistency, for example, drop size, drop size distribution, emulsions of a desired volume and/or containing 1 bead and 1 DNA template per aqueous droplet. It may also be desirable to provide an emulsion generation technique that yields substantially consistent emulsions over a range of different volumes.

### SUMMARY

According to various embodiments of the present teachings, a method of preparing an emulsion is provided. The method can comprise mixing together an aqueous phase solution, comprising a plurality of template beads, a library of templates from a sample, DNA polymerase, a buffer, dNTPs, one or more surfactants, and a pair of primers, to form a mixture. The mixture can then be contacted with an oil phase and then emulsified to form an emulsion comprising a plurality of microreactors. The emulsion can be disposed in a pouch, bag, or other flexible container, for example, transferred into a pouch or initially formed in a pouch. The emulsion in such a container is then subjected to conditions that enable a reaction, such as a polymerase chain reaction, to take place. The concentrations of the library (DNA sample molecule(s) and template beads, the total number of microreactors or droplets, and the average size of the microreactors, can be controlled such that, on average, about three to five out of every 10 to 13 microreactors contains one library molecule (a DNA sample) and at least one template bead. In some embodiments, the concentrations and sizes used can be such that there is very little chance of having two different DNA sample molecules attach to the same or a different template bead, in the same microreactor.

In some embodiments, the method can further comprise thermally cycling the emulsion to cause respective polymerase chain reactions in the microreactors. The polymerase chain reactions can cause the formation of a plurality of templated beads each comprising a plurality of amplicons of a respective template attached thereto. To collect the templated beads, the method can comprise breaking the emulsion to release the templated beads from the microreactors. Breaking the emulsion can comprise contacting the emulsion with an alcohol, for example, with propanol, butanol, or pentanol. The broken emulsion can then be subject to phase separation. In some embodiments, the templated beads can be denatured to form single-stranded templates attached thereto. According to various embodiments, the method can further comprise collecting the released templated beads, washing the collected templated beads, enriching the collected templated beads to form enriched beads, and/or eluting the enriched templated beads.

According to various embodiments, the method can comprise placing the pouch in a dual-sided thermocycler and thermally cycling the emulsion in the pouch. For example, the method can comprise subjecting the emulsion in the pouch to polymerase chain reaction using a thermal cycler and method as described, for example, in concurrently filed U.S. Pat. No. ______ to Liu et al., entitled “System Comprising Dual-Sided Thermal Cycler and Emulsion PCR in Pouch,” Attorney Docket No. 5010-480-02 which is incorporated herein in its entirety by reference.

According to various embodiments, the method can comprise enriching the templated beads using an enriching system and method as described, for example, in concurrently filed U.S. Pat. No. ______ to Karger et al., entitled “Column Enrichment of PCR Beads Comprising Tethered Amplicons,” Attorney Docket No. 5010-480-03, which is also incorporated herein in its entirety by reference.

According to various embodiments of the present teachings, a method of making a water-in-oil emulsion is provided that comprises: adding a volume of oil to a round wall container; spinning an impeller disposed in the round wall container in the volume of oil at a selected constant rpm, such that a stable vortex exists; adding a volume of aqueous solution to the stable vortex; and, after adding the volume of aqueous solution, continuing to spin the impeller in the combined volumes of oil and aqueous solution for a selected time period, thereby forming a water-in-oil emulsion in the round-wall container. The water-in-oil emulsion can be stable when thermocycled and has a selected drop size distribution.

The present teachings also provide a system that comprises an emulsion as described herein, in a pouch. The pouch can comprise a bag, a foil bag, a plastic bag, or another flexible container. The emulsion can comprise an aqueous phase and an oil phase as described herein, wherein the aqueous phase comprises a plurality of microreactors as described herein. In some embodiments, at least some of the microreactors contain a templated bead that comprises a plurality of amplicons of a respective one of the templates, attached thereto. In some embodiments, at least some of the microreactors are free of a templated bead that comprises a plurality of template amplicons attached thereto.

The pouch can comprise any suitable material, for example, that is flexible and exhibits good tear strength. The pouch can comprise a plastic material, an aluminum material, aluminum foil, polypropylene, a combination thereof, or the like. In some embodiments, the method can comprise heat-sealing the pouch after the emulsion is disposed therein.

In some cases, the pouch can comprise a plastic material, aluminum material, or a combination thereof, having a thickness of 20 mils or less, for example, 12 mils or less, or about 7 mils or less. To provide enhanced thermal conductivity, the pouch can comprise an aluminum foil layer, for example, having a thickness of 20 mils or less. In some embodiments, the pouch can comprise a plastic material and a heat-seal made by heat-sealing the pouch. In some embodiments, the pouch comprises a top, a bottom, and an openable and closeable port at the top, through which the emulsion can be loaded into the pouch, and through which the emulsion can be removed from the pouch after processing.

In some embodiments, the system can comprise an emulsion comprising microreactors have an average diameter size of from 3.0 to 20.0 micrometers (μm), for example, from 5.0 μm to 10.0 μm, or from 8.0 μm to 9.0 μm.

According to various embodiments a system is provided that comprises an emulsifier module, an amplifier module, and an enricher module, which together can be used to form templated beads useful in a bead-based DNA sequencing platform. In some embodiments, the system can comprise in-line filters to non-magnetically concentrate beads and perform buffer exchanges. In some embodiments, a dia-filtration unit and method can be used in lieu of a manual glycerol cushion and centrifugation. In some embodiments, beads are de-aggregated using sheer flow through a syringe valve.

### BRIEF DESCRIPTION OF THE DRAWINGS

The accompanying drawings, which are incorporated in and constitute a part of this specification, illustrate exemplary embodiments and together with the description, serve to explain various principles. The skilled artisan will understand that the drawings, described below, are for illustration purposes only. The drawings are not intended to limit the scope of the present teachings in any way.

FIG. 1 shows a templated bead workflow from emulsion generation to bead enrichment, according to various embodiments of the present teachings.

FIG. 2 is a flowchart showing exemplary process steps that can be carried out by a method and system according to various embodiments of the present teachings.

FIGS. 3A-3C illustrate three steps associated with an emulsion preparation and amplification method for sequencing applications, according to various embodiments of the present teachings.

FIG. 4 is a schematic drawing of a system useful in forming an emulsion in a round container, according to various embodiments of the present teachings.

FIGS. 5A-5B are microphotographs showing a phenotypic comparison between an emulsion prepared by an emulsion preparation system according to the present teachings, and an emulsion prepared using a conventional system.

FIGS. 6A-6H show various impeller configurations according to the present teachings, that provide stable emulsions of good average diameter size.

FIG. 7A depicts a paddle-blade configuration useful according to various embodiments of the present teachings.

FIG. 7B shows the shear profile generated by a paddle impeller having a displacement angle of 90°.

FIGS. 8A-8F and 9A-9H depict the effect of emulsion speed on drop size and size distribution, according to various embodiments of the present teachings.

FIG. 10 is a graph showing the effect of D/d ratio on drop size at major peaks for various emulsion preparations, according to various embodiments of the present teachings.

FIG. 11 illustrates plots of drop sizes at a major peak as a function of revolutions per minute, according to various embodiments of the present teachings.

FIG. 12 is a graph showing the reproducibility of a relatively large emulsion preparation, according to various embodiments of the present teachings.

FIGS. 13A-13H illustrate the robustness and utility achieved according to various embodiments of the present teachings.

FIGS. 14A-14C illustrate the adaptability and robustness of an emulsion preparation system according to various embodiments of the present teachings.

FIG. 15 is a graph showing the volume reduction of HIPE emulsions.

FIG. 16 depicts calculated volume for four groups of HIPE emulsions formed according to various embodiments of the present teachings.

FIG. 17 shows an exemplary emulsifier instrument according to various embodiments of the present teachings.

### DESCRIPTION

According to various embodiments of the present teachings, an emulsion is created that comprises droplets of an aqueous phase, or microreactors, in which clonal amplification takes place. Microreactors containing a single template bead and a single template, called monoclonal microreactors, are desired and can be formed according to the present teachings. Some microreactors, however, can be polyclonal such that they contain multiple templates, non-clonal such that they contain no template, or multi-bead-containing, and some microreactors exhibit a combination of these features.

After the emulsion is created, it can be thermally cycled to produce, for example, more than 30,000 copies of template amplified on to each template bead. Each template bead can comprise a respective primer, for example, a P1 primer, attached to a bead. In non-clonal microreactors, the template bead cannot amplify. Although beads are referred to often herein, it is to be understood that other template or target supports can be used, for example, particles, granules, rods, spheres, shells, combinations thereof, and the like. Furthermore, although the microreactors are described herein as containing components for PCR, it is to be understood that the microreactors can contain components for reactions other than PCR, for example, components for an isothermal reactions, components for another amplification reaction, components for an enzymatic reaction, or the like.

After emulsion PCR is complete, some of the template beads comprise amplicons of the template formed thereon, and are herein referred to as templated beads. Templated beads comprise template beads on which amplification took place in the respective microreactors. Some of the template beads do not comprise amplicons of the template formed thereon, and are herein referred to as non-templated beads. Non-templated beads comprise template beads on which no amplification took place in the respective microreactors. The non-templated beads can also be referred to as non-amplifying beads.

The emulsion can then be broken, for example, with 2-butanol, and the templated beads and non-templated beads can be recovered and washed. Enrichment can be performed to isolate template beads from non-templated beads. In some embodiments, an enrichment bead comprising a single-stranded P2 adaptor or P2 primer can be used to capture the templated beads. The mixture of enrichment beads, enrichment bead-templated bead complexes, and non-templated beads, can then be subject to filtration followed by elution to isolate the templated beads.

In some embodiments, each of the templated beads and each of the non-templated beads can have a diameter of from 0.25 μm to 2.0 μm, from 0.5 μm to 1.0 μm, from 0.9 μm to 1.2 μm, or from 0.7 μm to 1.1 μm. In some embodiments, the one or more enrichment beads can each have a diameter, or collectively an average diameter, of from 3.0 μm to 20 μm, for example, from 5.0 μm to 15 μm, from 6.0 μm to 10 μm, or from 6.4 μm to 6.8 μm.

Reference will now be made in detail to various exemplary embodiments, examples of which are illustrated in the accompanying drawings. Wherever possible, the same reference numbers will be used throughout the drawings to refer to the same or like parts.

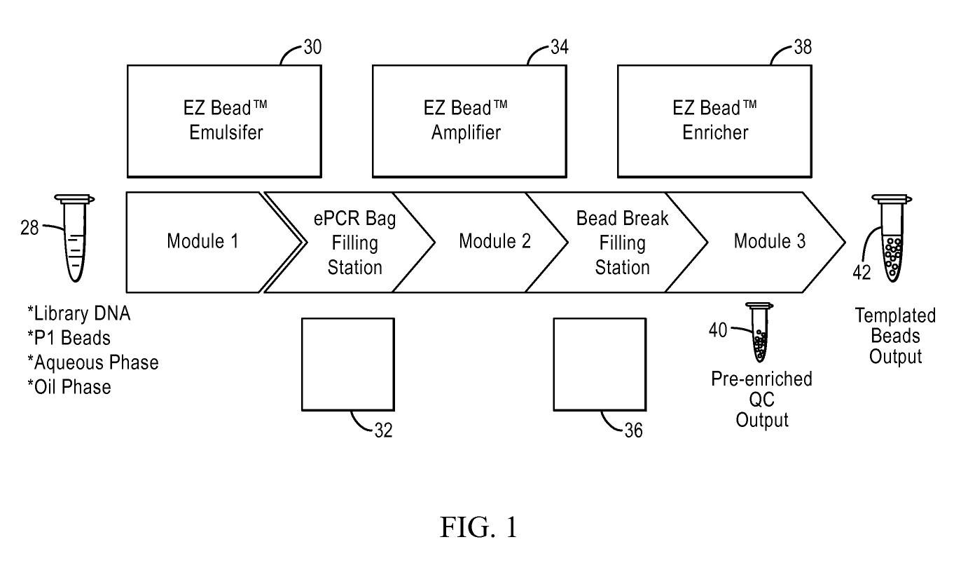

FIG. 1 shows a templated bead workflow from emulsion generation to bead enrichment, according to various embodiments of the present teachings. FIG. 1 shows an exemplary process workflow and the system components for carrying out the process. An input 28 to be processed by the system can comprise an aqueous phase master mix, an oil phase master mix, template beads, and a collection or library of templates such as DNA sample molecules from the same or from different samples. The aqueous phase master mix can comprise water, dNTPs, and DNA polymerase. The various components for the emulsion can be brought together and emulsified in an emulsifier module 30 during a first step of the five-step process depicted. Emulsifier module 30 is also referred to herein as module 1 in the process flow diagram shown in FIG. 1. After forming an emulsion using emulsifier module 30, the mixture can be poured into a pouch using an ePCR pouch filling station 32. After filling, the pouch can be closed or sealed, for example, by heat-sealing. The pouch and its contents can then be thermally cycled using an amplification module 34 that is also referred to herein as module 2 in the prOcess flow diagram shown in FIG. 1. Following thermocycling using amplification module 34, the contents of the pouch can be poured into a bead break vessel at a bead break filling station 36 to carry out a fourth step of the five-step process.

After breaking the emulsion to release the beads, the beads can be enriched using a bead enrichment module 38 that is also referred to herein as module 3 in the process flow diagram shown in FIG. 1. The beads can comprise productive beads, referred to herein as templated beads. Templated beads can comprise beads that have undergone a desired reaction, for example, upon the surface of which multiple reactions have taken place. The beads can also comprise non-templated beads, which were not productive.

According to various embodiments, there can be two or more outputs of the system, including, for example, a first output 40 that includes a pre-enriched quality control output that can provide a user with information on bead clonality. A second output 42 can be provided that includes templated beads that are ready for terminal transferase modification, deposition on a slide or in a flow cell, a combination thereof, or the like.

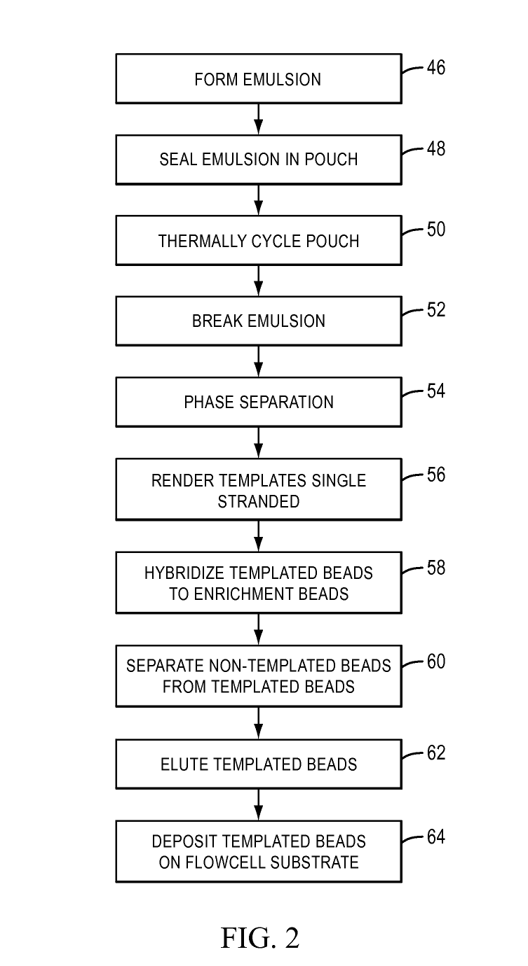

While the system described in connection with FIG. 1 comprises five different modules and stations, and five process steps, it is to be understood that the system can comprise less or more modules and/or stations and that various modules and/or stations can be combined together. Furthermore, it is to be understood that the method can comprise fewer or more than five steps and that the five exemplary steps described in connection with FIG. 1 can each independently be omitted or combined with one or more other steps. In some embodiments, other amplification reactions, isothermal amplification reactions, enzymatic reactions, biological reactions, and the like, can be carried out instead of or in addition to a polymerase chain reaction. Moreover, additional steps can be provided in the method as exemplified with reference to FIG. 2.

FIG. 2 is a process flow diagram showing various process steps associated with a method according to various embodiments of the present teachings. As with FIG. 1, the process steps shown in FIG. 2 can each independently be omitted, substituted, or combined with one or more other process steps. As shown in FIG. 2, a first step 46 of the method can comprise forming an emulsion. The emulsion can be formed according to any of the various embodiments of the present teachings and as described herein. In a next step 48, the emulsion is sealed in a pouch. In an exemplary embodiment, the pouch can comprise a heat-sealable material and the sealing can comprise heat sealing the emulsion in the pouch. The sealed pouch can then be thermally cycled as depicted by process step 50.

In an exemplary embodiment, a dual-sided thermal cycler is used to thermally cycle the emulsion in the pouch. The thermal cycling can result in templated beads each comprising amplicons of a respective template and tethered or hybridized to a primer pre-deposited on a surface of a respective template bead. The method can further comprise an emulsion breaking step 52 followed by a phase separation step 54, tailored to separate the templated beads from the remainder of the emulsion. A denaturing step 56 can be provided to render the templates tethered to the templated beads, single stranded.

Templated beads bearing the single-stranded templates can be hybridized to enrichment beads to form a capture complex, as depicted at step 58. In the next step, the templated beads captured in the capture complexes can be separated from non-templated beads in a separation step 60, for example, using a size-exclusion technique. In a next step 62, the templated beads are eluded from the capture complexes and are collected. Subsequently, the collected templated beads can be deposited on a flow cell substrate or otherwise formed into an array in a flow cell.

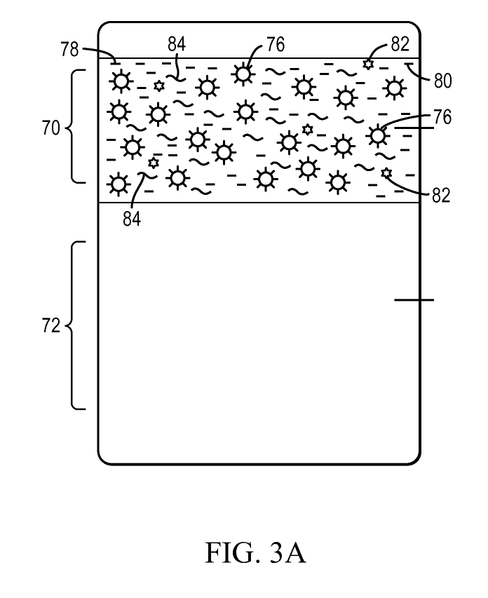

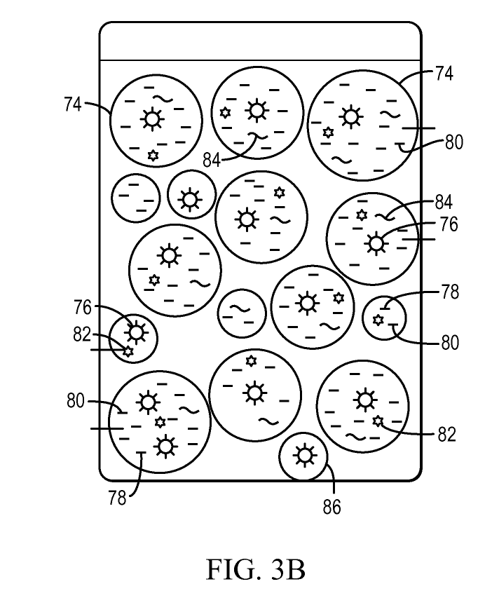

FIGS. 3A-3C illustrate the general steps associated with emulsion preparation and amplification for sequencing applications, according to various embodiments of the present teaching. The emulsion can comprise an aqueous phase 70 and an oil phase 72 wherein aqueous phase 70 comprises constituents useful for amplifying DNA templates, for example, a library of templates from a single sample. In some embodiments, the emulsion comprises clonal or monoclonal reactors or microreactors 74 containing a single DNA template molecule. Some sequencing platforms, for example, the SOLiD sequencing system by Applied Biosystems, Foster City, Calif., utilize emulsion polymerase chain reaction (ePCR) approaches that provide compartmentalization of PCR reactions in discrete aqueous droplets of an inverse emulsion such as a water-in-oil (W/O) emulsion. In some embodiments, a template bead 76, approximately 1 μM in diameter, and comprising surface-immobilized oligo nucleotides, can be entrapped in each discrete aqueous droplet microreactor 74. Each microreactor can also contain PCR reagents such as a forward primer 78, a reverse primer 80, a DNA polymerase 82, and a single DNA sample molecule 84.

In some cases, some of the microreactors can comprise some of the components but not others. For example, microreactor 86 contains no template and no DNA polymerase, and would not be expected to yield a templated bead. According to various embodiments, the microreactors can contain other components for reactions other than PCR, for example, components for an isothermal amplification, components for another amplification reaction, components for an enzymatic reaction, components for a ligation reaction, or the like.

In various embodiments, the emulsion is thermally cycled from approximately 64° C. to 96° C. for 40 or 60 cycles (depending on the length of the template molecule being used). Subjecting the microreactors to PCR conditions in this manner results in clonal amplification yielding a product that is composed of a singular DNA species. The amplification conditions can cause a templated bead 88 (FIG. 3C) to be formed in many of microreactors 74. Concentrations of components can be used to minimize the number of microreactors 90 containing two or more templated beads. As is depicted, the microreactors can include microreactors 92 that contained no template molecule or no template bead and thus do not produce a templated bead. After amplification, the amplified products are then subjected to subsequent downstream processing, including emulsion breaking, bead enrichment, array deposition of beads, and sequencing.

The emulsion preparation apparatus of the present teachings can be adapted to readily prepare a wide range of different emulsion volumes, for example, of from approximately 5 mL to 250 mL or more, without maintaining a stock of differently sized or configured consumables to accommodate a particular emulsion volume. The emulsion exhibit small drop size variation, a slow rate of reversion or phase separation, and an adaptability to a wide variety of volume sizes. Additionally, the emulsion preparation apparatus of the present teachings is cost-effective, user-friendly, and robust, and provides a reproducible means to prepare inverse emulsions for ePCR.

In some embodiments, the present teachings provide devices, methods, and formulations for the preparation of inverse (water-in-oil) emulsions for polymerase chain reactions. In various embodiments, the discrete aqueous phase (droplets) can entrap a particle, for example, a magnetic particle of about 1 μM diameter size and having oligonucleotides immobilized on its surface. The discrete aqueous phase droplet can also comprise PCR reagents such as dNTPs, enzymes, co-enzymes, salts, buffers, surfactants, and a template molecule such as a DNA sample. The template molecule can be a sample DNA molecule, for example, a template from a library of templates from a single sample. The continuous phase can comprise oil with or without an added surfactants that have hydrophilic-lipophilic-balances (HLB) values equal to or less than 5.0 and below. According to various embodiment of the invention, the surfactants can be a mixture of surfactants having various HLB values. For those who are skilled in the art can appreciate that the surfactant affinity different (SAD) of an oil phase can be adjusted by using various surfactants with various HLB values such that a stable inverse (water-in-oil) emulsion can be prepared.

The liquid oil phase can comprise a mineral oil such as Petroleum Special, an alkane such as heptadecane, a halogenated alkane such as bromohexadecane, an alkylarene, a halogenated alkyarene, an ether, or an ester having a boiling temperature above 100° C. The oil phase can be insoluble or slightly soluble in water. The ratio between the continuous oil phase and the discrete aqueous phase may range from 1/0.1 v/v to 4/1 v/v, from 0.5/1 to 3/1, from 0.8/1 to 1/1, or as desired.

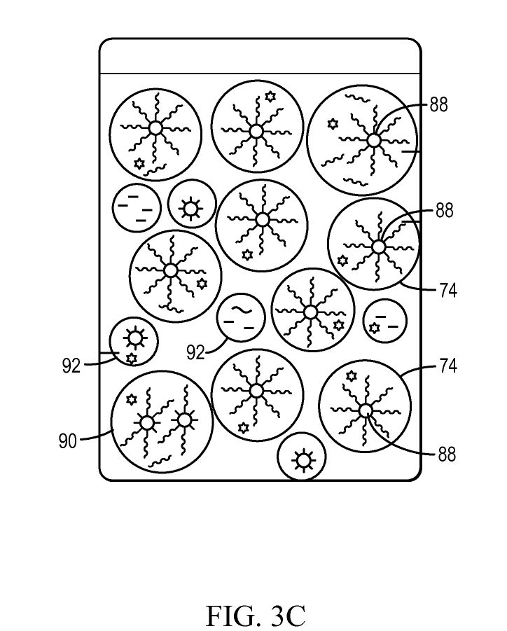

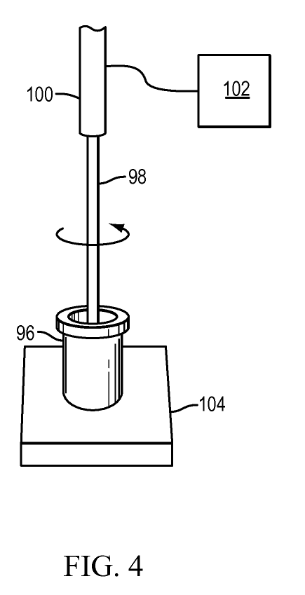

FIG. 4 illustrates the components of an exemplary emulsion preparation device according to various embodiments of the present teachings. The device comprises a mixing chamber 96, an impeller shaft 98, an impeller (not shown), an electrical motor 100, and a controller 102. Mixing the chamber 96 can be held by friction, gravity, a clamp, a biasing device, or the like, onto a platform 104. Electric motor 100 can be configured to drive impeller shaft 98 in the direction shown by the arrow, and controller 102 can be configured to control the speed of rotation. In some embodiments, the system benefits from a combination of the impeller design and the usage parameters associated with the impeller for agitating the water and oil phases. The present teachings disclose impeller designs and usage parameters that generate emulsions with desired characteristics. Novel characteristics of the impeller design can include, but are not limited to, the geometry and/or dimensions of the impeller, the position of the impeller within a mixing chamber (for example centered or off-centered), the optimized ratio between the internal diameter of the mixing chamber and the diameter of the impeller (D/d ratio), the optimized ratio between the sample liquid height (aqueous and non-aqueous liquids used in creating the emulsion) and the impeller height to diameter ratio (H/d ratio), the optimized ratio between the liquid height and the depth of the impeller (H/h ratio), and the impeller speed (rpm) for generating the emulsion. These parameters can be adjusted to generate a wide range of stable water-in-oil emulsions, for example, of from approximately 5 mL to 250 mL or more, with controllable and reproducible drop size and drop size distribution.

As shown in FIG. 4, various configurations of an exemplary emulsification system are provided according to embodiments of the present teachings. In some embodiments, the emulsification apparatus can further comprise a dispensing tube located in a generally fixed position. The dispensing tube can act as a conduit for metering in the aqueous phase during emulsification. In various embodiments, the emulsification apparatus platform 104 can comprise a movable platform having an adjustable height. It will be appreciated that motor 100 used to drive the impeller can be in various forms, configurations and sizes and can have a variable or controllable speed (rpm) profile in clockwise or counter-clockwise rotation.

In various embodiments an emulsifying chamber employed in connection with the present teachings may be a cost-effective, disposable plastic bottle or container, for example, polypropylene vessel made by Taral Plastics and supplied by VWR. Polypropylene vessels of this sort are available in a variety of different sizes and may be obtained with capacities of 60-, 120-, 250- and 500-mL. Such vessels may contain the water and oil phases and have a dimensionality to accommodate the impeller to prepare emulsions (for example from approximately 6.0 mL/batch to 233.0 mL/batch or more).

In various embodiments, an emulsion with a controlled drop size and size distribution can be prepared from the vortex generated by the impeller. In some cases, the vortex can be symmetric and stable as opposed to swirling or bouncing around. In various embodiments, impeller shaft 98 and emulsification vessel 96 are centered within an indentation in the top surface of the platform, to keep the vessel in a generally fixed position.

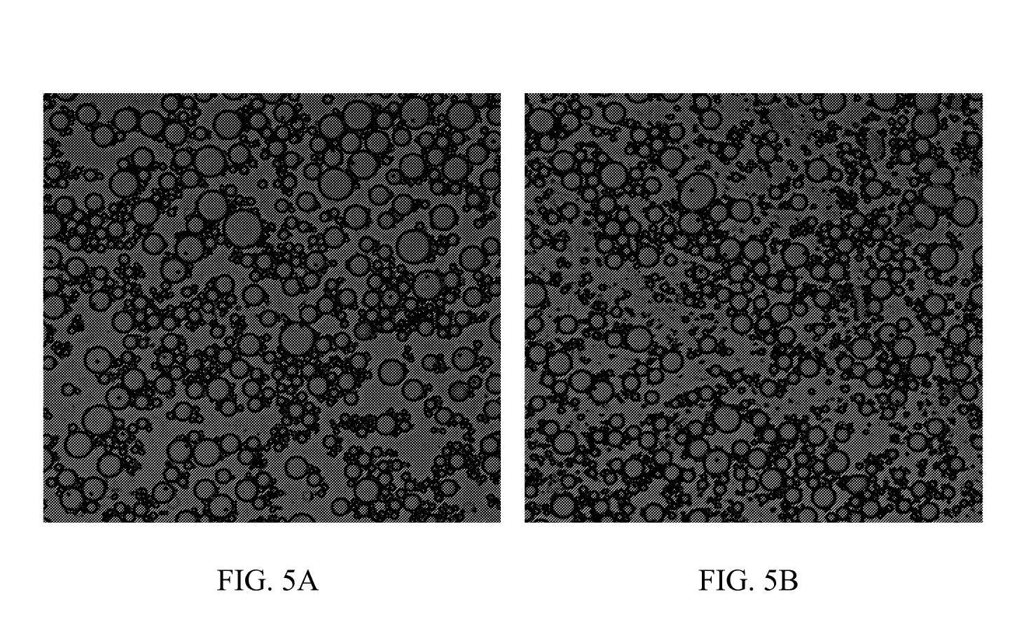

It will be appreciated that the properties of some emulsions are such that the emulsion is inherently unstable such that the discrete droplets coalesce to form bigger droplets over time, and the emulsion eventually undergoes phase separation. Factors that affect the stability of an emulsion include drop size, drop size distribution, surfactant properties, surfactant concentration, oil and water viscosity ratio, aqueous phase ionic strength, temperature, and mechanical actions taken in preparation of the emulsion. FIGS. 5A-5B illustrate an exemplary and non-limiting phenotypic comparison of an emulsion prepared according to the present teachings (FIG. 5A) versus an emulsion prepared via conventional methods (FIG. 5B).

In general, emulsions with drop sizes smaller than 1 μM are typically more stable than those with droplets greater than 10 μM. The so-called Oswald Ripening Effect in which small droplets become smaller and big droplets become bigger droplets helps to explain why an emulsion with large drop size distribution is susceptible to phase separation. According to various embodiments of the present teachings, an emulsion having a relatively narrow drop size distribution is generated to obtain a reasonably stable emulsion capable of enduring through repeated steps of thermal cycling in PCR. The present emulsions can remain stable for multiple cycles over a temperature range from 64° C. to 95° C.

Some surfactants used in an inverse emulsion preparation are insoluble or sparingly soluble in the discrete aqueous phase but soluble in the oil phase. In such cases, the surfactant concentration can be relatively higher than the critical concentration desired to partition at the interfaces. In this regard, a particular surfactant can be chosen based on its effectiveness in preventing coalescence. For example, a polymeric surfactant effective in preventing two droplets from merging due to steric effects can be used in some embodiments.

Additionally, it will be appreciated that the effect of viscosity/density ratios between the two phases can be significant. Based on the Stokes Law of sedimentation, a viscosity/density ratio of 1:1 may tend to favor emulsion stability. Furthermore, the mechanical means employed for the preparation of emulsions can be a principle contributing factor to the ultimate drop size, drop size distribution and emulsion stability and so impeller design and speed can be considered in determining an appropriate configuration.

### Emulsifier and Emulsifying Chamber

One type of conventional apparatus used for emulsion generation is a homogenizer such as the IKA ULTRA-TURRAX DT-20 or DT-50. These devices comprise one stationary wheel and one concentric rotating wheel in an emulsifying chamber. The dimensions of the wheels, the chamber, and the positions of the wheels are typically fixed. In various embodiments the emulsifier of the present teachings, the impeller is driven by an electrical motor and the design, geometry, and position of the impeller in respect to the emulsifying chamber can be configured and adjusted as desired to give optimal effects on an emulsion in terms of its drop size and drop size distribution. Such an apparatus provides a more flexible and configurable apparatus that can be adjusted to accommodate a variety of different emulsion preparations having desirable properties and volumes.

In various embodiments, the emulsion can comprise a suspension of small discrete droplets of a disperse phase in a continuous phase. In an inverse emulsion, the discrete phase is an aqueous solution and the continuous phase is oil. In preparing an emulsion, a shear force is utilized to break down the disperse phase into small droplets. A spinning impeller designed according to the present teachings can generate such a shear force. According to the so called Taylor Factor, a larger droplet may break down into two smaller droplets under a shear force if the elongated droplet has a length to width ratio greater than approximately 3 to 1, as described in G. I. Taylor, Proc. Royal Society 1934, 29, 501; (b) G. I. Taylor, Royal Society of London 1932, 41, which is incorporated herein in its entirety by reference. It is understood according to the present teachings that emulsions with relatively small droplets can be obtained under a shear force of sufficient strength. While a conventional spinning magnetic stir bar can be used to prepare certain emulsions, as described in D. Dressman, et al, PNAS 2003, 100 (15), 8817-8822, which is incorporated herein in its entirety by reference, such an approach may not be desirable in the presence of magnetic particles that can be attracted to or become attached to the magnetic stir bar. Furthermore, in such conventional designs a magnetic stir bar is placed in direct contact with the bottom of the mixing chamber and, for example, there is no optimal H/h ratio because h is zero. At high rpm, the rotation of the stir bar may cause an undesirable mechanical grinding action which can damage the magnetic beads to be contained within the emulsion.

Literature reports for the shear profiles of conventional spinning impellers may produce undesirable emulsions that are not necessarily homogeneous, as described in, for example, M. T. Stillwell, et al, Ind. Eng. Chem. Res. 2007, 46, 965-972 and S. R. Kosvintsev et al, Ind Eng. Chem. Res. 2005, 44, 9323-9330, which are incorporated herein in their entireties by reference. As a result, an emulsion prepared under such conditions can exhibit an undesirably broad drop size distribution and often more than one population of drop size as well. For example, emulsions prepared by chaotic vortexing of a 50-mL conical FALCON tube can exhibit multiple populations in drop size distribution. Impellers of various designs that are suitable for mixing may not be suitable for preparing emulsions with narrow drop size distributions.

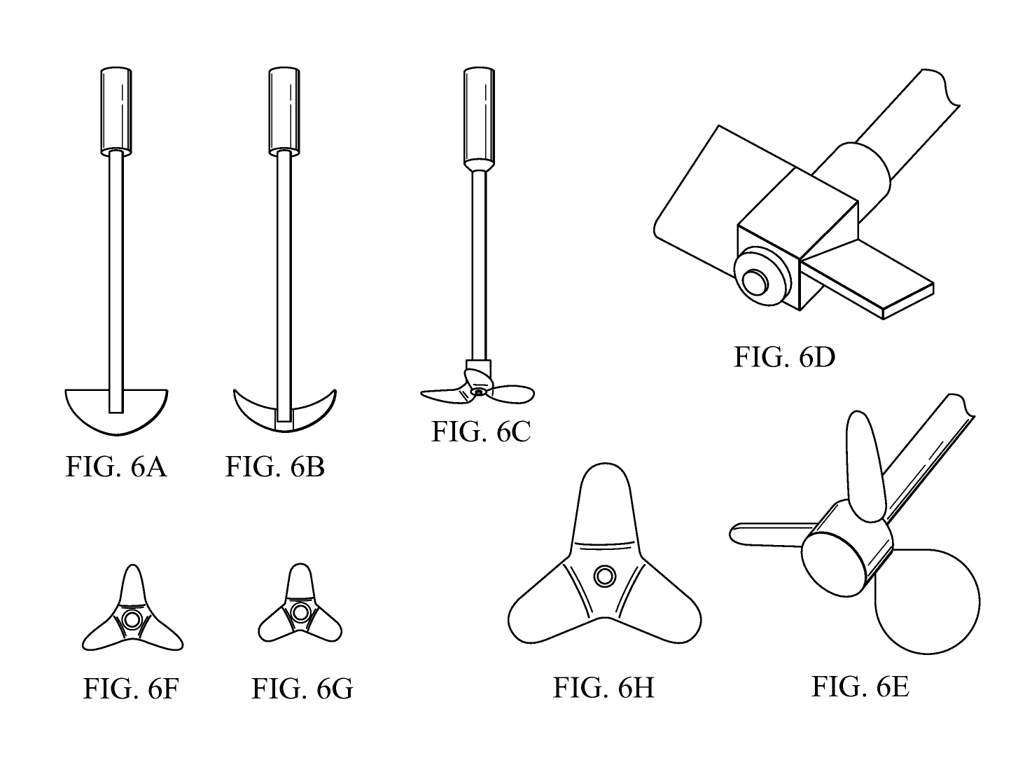

According to the present teachings, impeller designs such as those depicted in FIGS. 6A-6H can be used in systems configured to provide stable emulsions with relatively narrow drop size distribution characteristics. In various embodiments, the displacement angle of the blade of the impeller can be from 5° to 35°, for example, from 10° to 30° or from 15° to 25° such as exemplified by the impellers shown in FIGS. 6C, 6F, 6G, and 6H. In some embodiments, the displacement angle of the blade can be from about 40° to about 50°, or about 45°, such as shown by the impellers of FIGS. 6D and 6E. In some embodiments, the displacement angle of the blade can be from about 80° to 100°, or from 85° to 95°, or about 90° as shown in FIGS. 6A and 6B. In some embodiments, the impeller can comprise a tri-lobe configuration, for example, akin to a tri-lobe arrangement of propeller blades on a propeller.

Parameters that Affect prop Size and Size Distribution

As discussed previously, the characteristics of a particular emulsion can depend on shear force imparted by the impeller. Drop size and size distribution of an emulsion therefore can depend on the shear profile within the emulsifying chamber or vessel. In various embodiments, the shear profile can be controlled by parameters such as the ratio between the internal diameter of the emulsifying chamber and the diameter of the impeller (D/d ratio); the ratio between the liquid height and the diameter of the impeller (H/d ratio); the ratio between the liquid height and the height of the impeller above the bottom of the emulsifying chamber (H/h ratio); the displacement angle of the impeller blades; and the spinning speed (rpm) of the impeller.

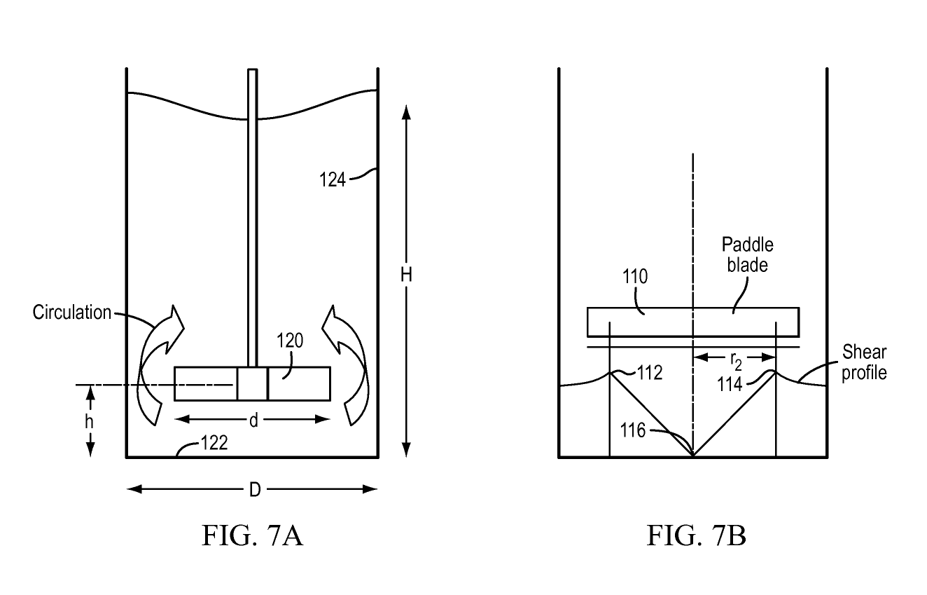

As shown in the illustration in FIG. 7B, the shear profile generated by spinning a paddle impeller 110 having a displacement angle of 90° may not be homogeneous, as described, for example, in M. T. Stillwell, et al, Ind. Eng. Chem. Res. 2007, 46, 965-972 and S. R. Kosvintsev et al, Ind. Eng. Chem. Res. 2005, 44, 9323-9330, which is incorporated herein in its entirety by reference. In general, such an impeller configuration has two maxima 112 and 114 close to the ends of the blades and there is a reduced or near-zero shear 116 toward the center underneath impeller 110. Consequently, an emulsion generated near the ends of the blades around the maxima would have relatively small drop size as compared with those at the center of the impeller and around the walls of the chamber. This impeller configuration therefore may results in a broad distribution in drop size.

In various embodiments of the present teachings as shown in FIG. 7A, the depth (h) of impeller 120 can be configured to be relatively small in order to help flatten the shear profile. Such a configuration may aid in minimizing the maxima. In an example, depth h can be from 0.1 to 5 mm above a bottom 122 of an emulsifying chamber 124, for example, from 1.0 mm to 4.0 nun or from 1.5 mm to 3.5 mm. The illustration shown in FIG. 7A depicts a paddle-blade configuration for circulation/convection of a liquid during emulsification. The extent of circulation affects the efficiency of emulsification which in turn attributes to the drop size and drop size distribution. In various embodiments of the present teachings, the D/d ratio may be in the range of from 1.1:1 to 3.0:1, for example, from 1.5:1 to 2.5:1, from 1.4:1 to 2.5:1, or about 2.0:1. The effectiveness of circulation may also depend on: the rotational or spinning rpm of the impeller; the impeller blade displacement angle; the height of the liquid (H); and the ratios H/h and D/H. In various embodiments, the D/H ratio ranges from 1:1 to 8:1, for example, from 2:1 to 6:1, or from 2:1 to 4:1. In various embodiments, the H/h ratio ranges from 10:1 to 2:1, for example, from 8:1 to 3:1, or from 6:1 to 4:1. In other embodiments where the viscosity of the emulsion is high, or the emulsion is a high internal phase emulsion (HIPE) exhibiting a high v/v ratio of aqueous discrete phase to oil phase, a relatively high D/H ratio can be used, for example, a D/H ratio of from 3:1 to 6:1 or from 4:1 to 5:1.

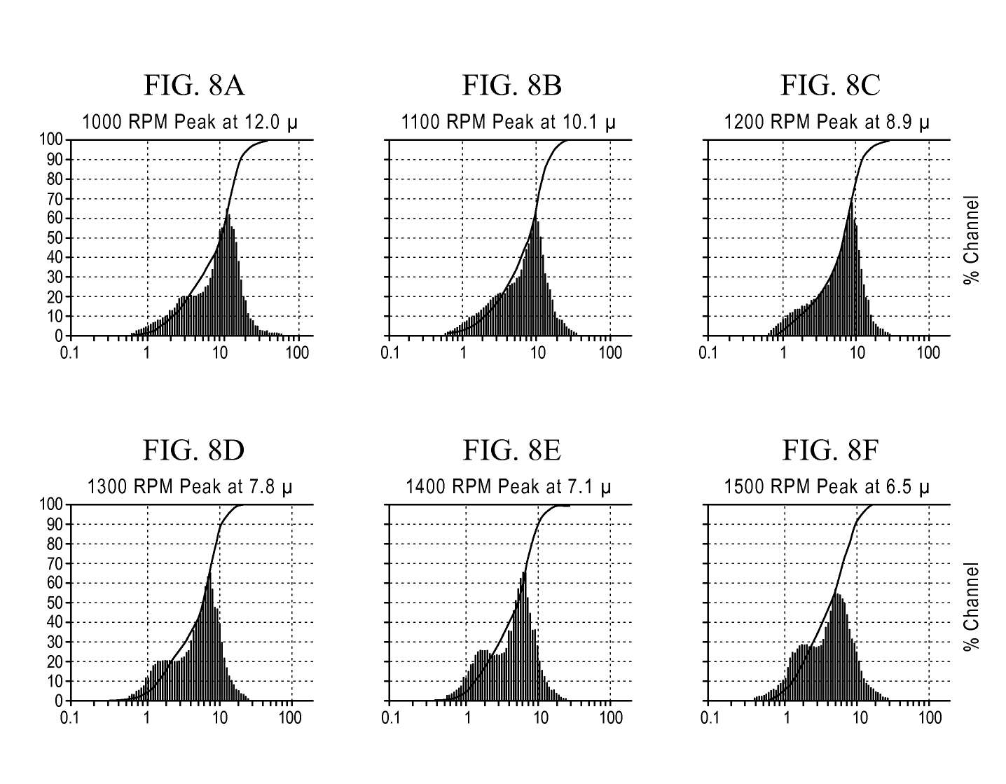

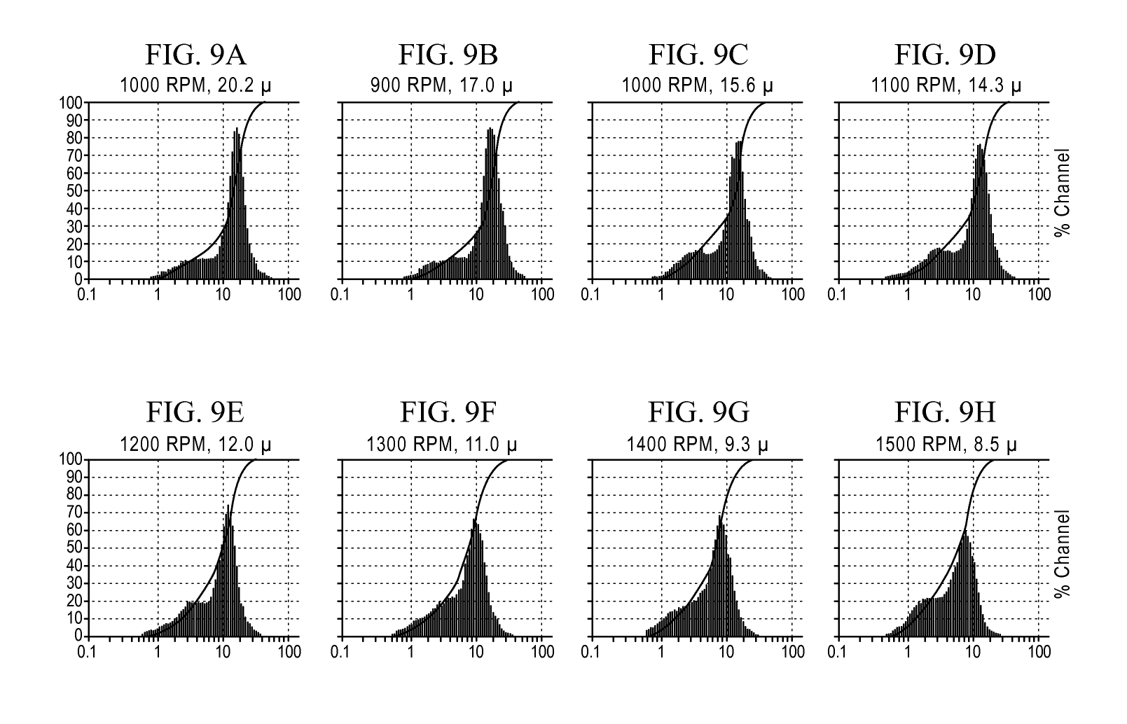

FIGS. 8A-8F and 9A-9H depict the effect of emulsification speed (rpm) on drop size and size distribution. Drop size distribution of the depicted emulsions result from emulsions prepared using about 9 mL of mineral oil containing a selected percentage of surfactant, in combination with about 5.6 mL of aqueous phase (without beads) and prepared in a glass emulsification chamber to generate a D/d ratio of about 1.6. It was observed that the size of the major peak decreased as the rpm increased. As shown in FIGS. 9A-9H drops size distribution of emulsions varies using similar phase preparations as described above but with a polypropylene vessel as an emulsification chamber and a D/d ratio of about 2.0.

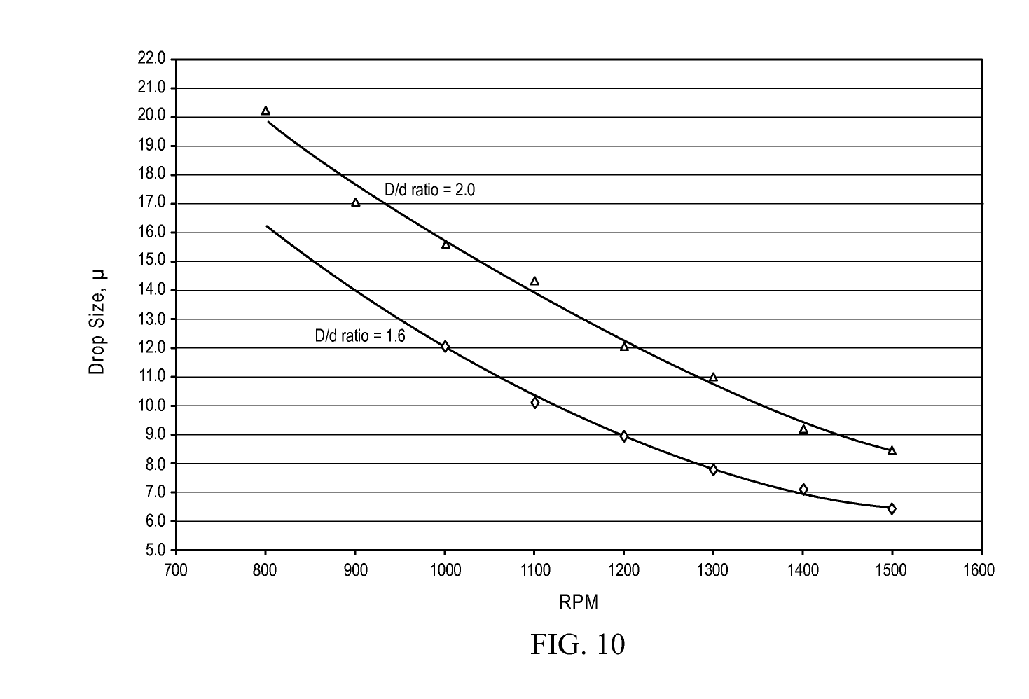

FIG. 10 depicts the effect of D/d ratio on drop size at the major peaks for emulsion preparations exemplified above. At a given rpm, the increase of D/d ratio was followed by an increase of drop size at the major peak.

According to the data presented above it will be appreciated that the impeller configurations of the present teachings are capable of reproducibly providing emulsion preparations with desired target drop sizes and size distributions.

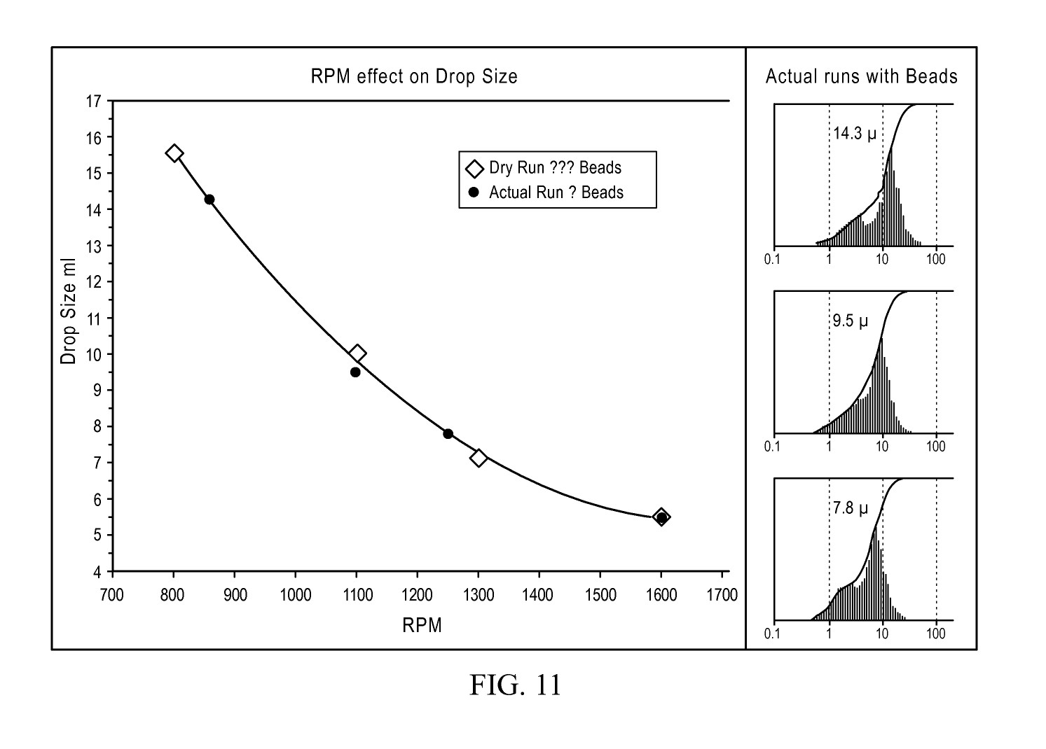

FIG. 11 illustrates plots of drop sizes at the major peak as a function of rpm. In this illustration, the open diamonds depict a calibration curve generated from 4 emulsions prepared using a 24 nun 3-blade impeller with a D/d ratio of 1.6 and an emulsion preparation comprising about 9 mL of mineral oil containing a selected percentage of surfactants, and about 5.6 mL of aqueous phase without magnetic beads. Using the calibration plot as a guide, the rpm useful to prepare various emulsions containing beads in their aqueous phase can be estimated. In the Figure, solid circles represent the approximate drop size at major peak for the exemplary emulsions which reside in close proximity to the calibration curve.

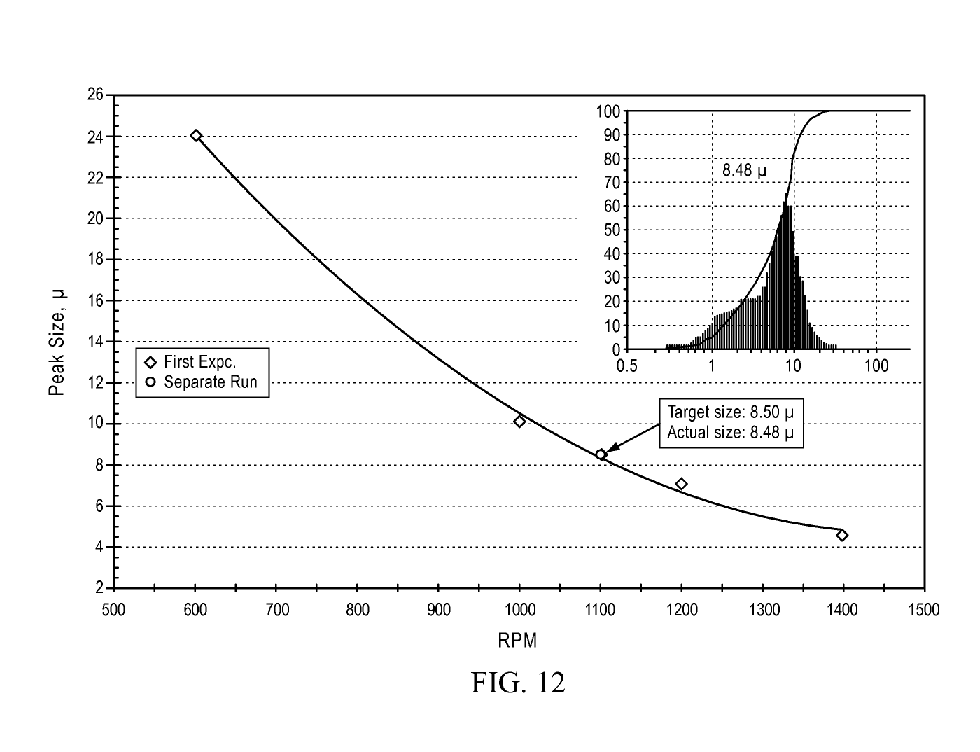

FIG. 12 depicts the reproducibility of a relatively large emulsion preparation of approximately 58 mL. Such an emulsion may be prepared with using a 33-mm half-moon impeller as depicted in FIG. 6A where the D/d ratio is approximately 1.4. An aliquot of approximately 22 mL of an aqueous solution containing MgCl₂ was added into approximately 36 mL of stirring mineral oil containing a selected percentage of surfactant. The solid diamonds depict the approximate drop size at the major peak of individual emulsions generated at various speeds (rpm). Based on this calibration curve, a separate run was conducted to target an approximate 8.50 μm drop size at the major peak by adjusting the impeller speed to 1100 rpm. The resulting emulsion, as depicted by the solid circle in FIG. 12, exhibited drop size at approximately 8.48 μm. These results clearly demonstrate the versatility and reproducibility of the emulsion preparation apparatus of the present teachings.

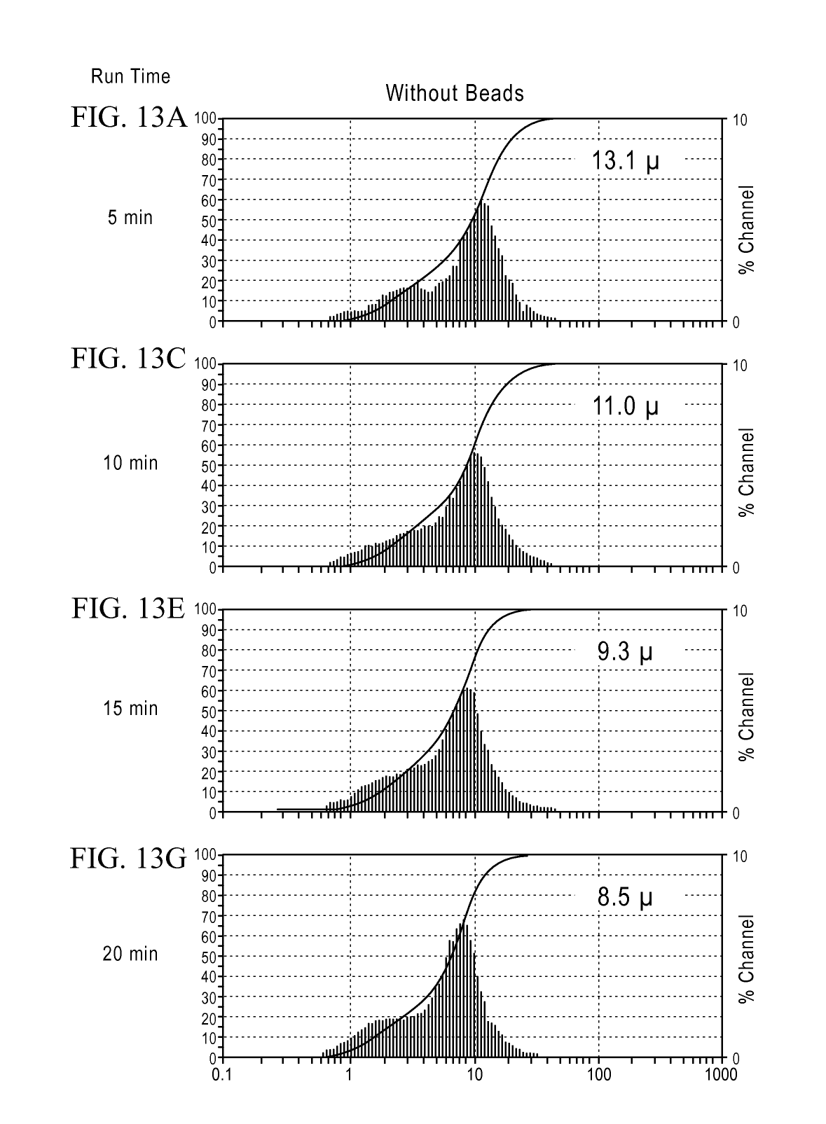

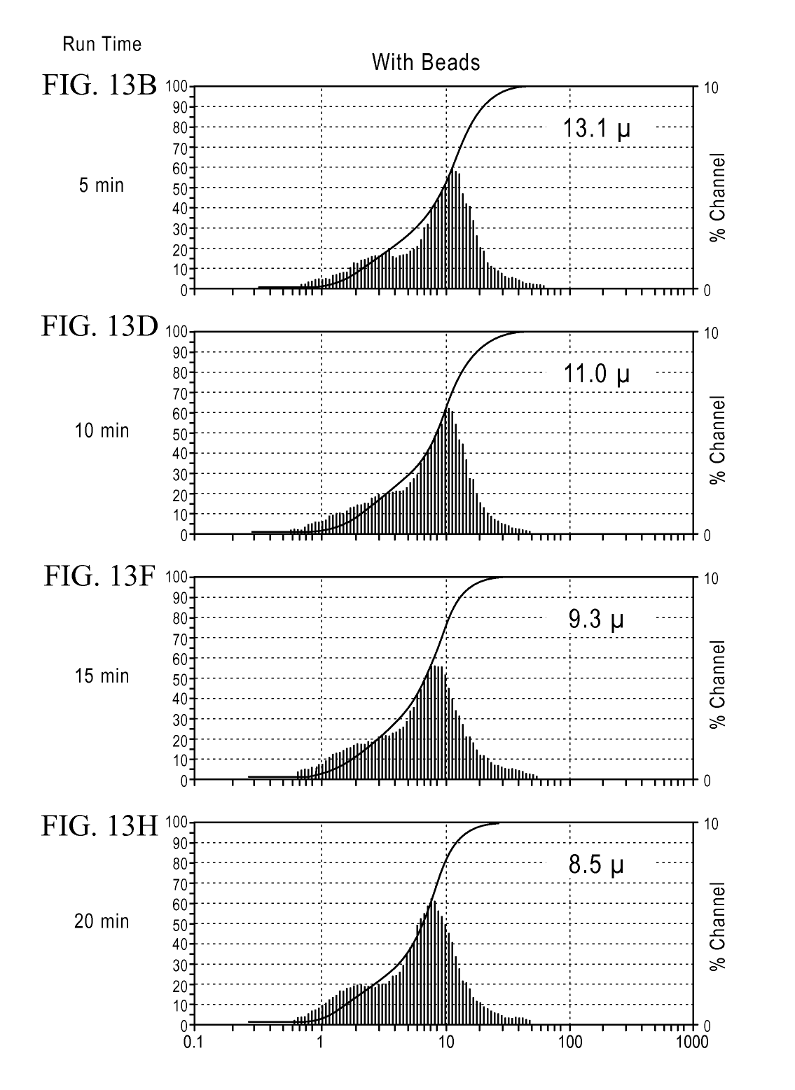

FIGS. 13A-13H further illustrates the robustness and utility achieved according to various embodiments of the present teachings. Here, data for two 58.4 mL emulsions was prepared with oil and aqueous phases, one with magnetic beads and the other without. Aliquot samples were taken at 5, 10, 15, and 20 minutes for drop size analysis. As depicted in FIGS. 13A-13H, the drop size and major peak and size distribution are very similar if not virtually identical indicating the reproducibility of emulsions generated using the apparatus.

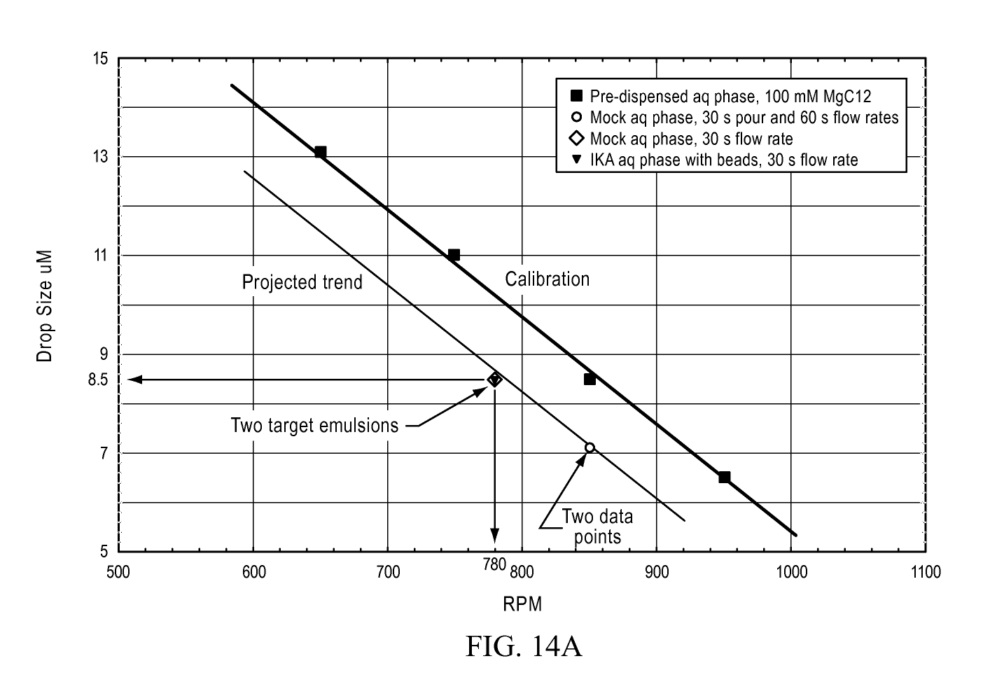

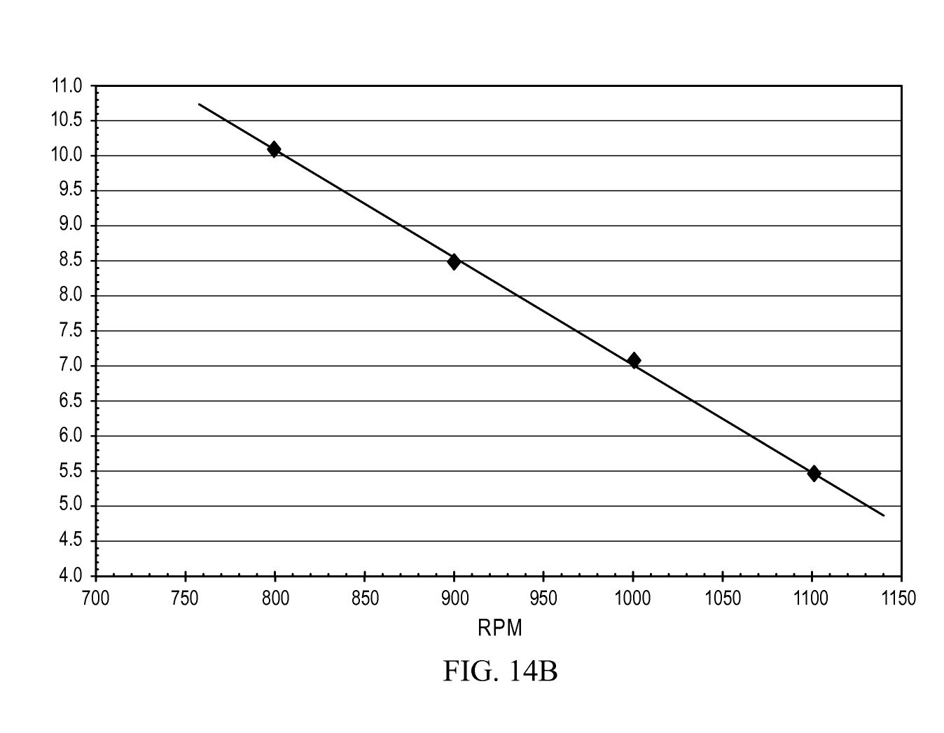

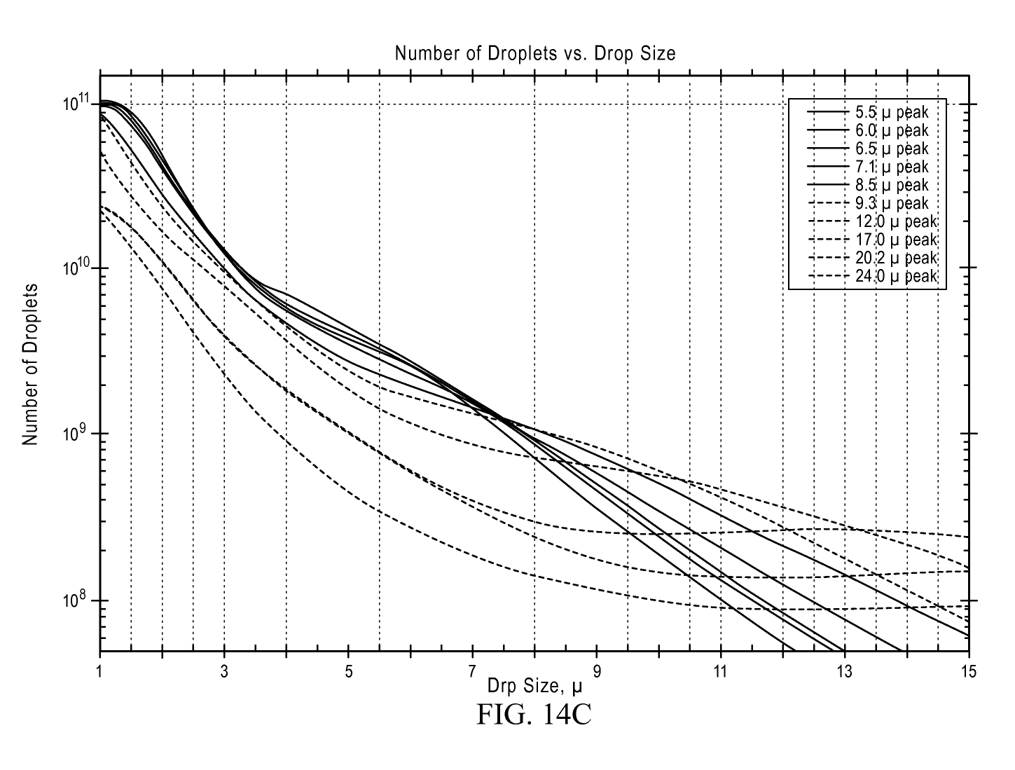

FIGS. 14A-14C further illustrate the adaptability and robustness of an emulsion preparation system according to various embodiments of the present teachings. In FIG. 14A, emulsions in batch sizes of approximately 117 mL were prepared with a 54-mm, 3-bladed impeller at a D/d ratio of 1.5, and polypropylene vessels as the emulsifying chambers. The linear calibration plot with solid squares was generated by emulsifying approximately 45 mL of aqueous MgCl₂ solution (the discrete phase) into approximately 72 mL of mineral oil containing a selected percentage of surfactants. Two separate mock emulsions were prepared with mock aqueous phase, that is, with aqueous phases free of magnetic beads, at 850 rpm. The drops sizes at major peaks of these two emulsions superimposed at approximately 7.10 μm. In order to estimate the rpm for an emulsion having a target drop size of 8.5 μm, a straight line parallel to the calibration line and passing through the two data points generated by the two mock emulsions can be drawn. The estimated emulsification speed for preparing such an IKA emulsion using an IKA aqueous phase comprising dNTPs, PCR polymerase, two primers, a DNA library, buffer, MgCl₂ and template beads can be estimated to be approximately 780 rpm. Two 117-mL emulsions were subsequently prepared at 780 rpm. Their peak drop sizes superimposed at 8.45 μm. Using a similar strategy, emulsions with an approximate batch size of 234 mL can be prepared.

FIG. 14B illustrates a comparison of drop size versus rotational speed of the impeller for which a generally linear response is observed, thus providing an efficient and reproducible means by which to generate an emulsion. FIG. 14C shows an evaluation of the drop population characteristics where the Y-axis reflects the drop population number and the X-axis reflects the drop size. Graphs for emulsion with varying centers of distribution as measured by the MicroTrac drop size analyzer are shown. This data demonstrates that over the range of approximately 1 to 10 microns (μm), there is minimal difference in drop population characteristics for emulsions with drop size at peak ranging from about 5.5 microns to about 12.0 microns. Consequently, it will be appreciated that the methods and apparatus of the present teachings provide robust and reproducible emulsions with minimal variations.

### Volume Considerations

In one aspect, the versatility of the present teachings allows the preparation of emulsions with batch sizes of varying sizes, for example, ranging from about 15 mL to about 234 mL. It will be appreciated however that larger and smaller emulsion volumes may be readily prepared and thus such volumes are not considered limiting upon the scope of the present teachings. Large volume emulsions are readily and conveniently produced according to the present teachings and are a notable improvement in comparison to using a conventional microtiter tray having 96-wells. Typically, a 96-well tray used for PCR thermal cycling can only accommodate approximately 14.6 mL of emulsion per plate and may require a substantially number of liquid transfer operations in order to prepare the emulsion plate.

In various embodiments it may be desirable to reduce the final volume of an emulsion while keeping the number magnetic beads, the number of droplets (reactors), drop size, and size distribution unchanged. Current conventional emulsion formulations for sequencing applications have an arbitrary volume ratio of approximately 0.6 to 1 for aqueous phase to oil phase. Literature observations regarding emulsions, especially the high internal phase emulsions, HIPE, suggest that a volume ratio of 6 to 1, or 9 to 1, for discrete phase to continuous phase, may be desirable, for example, as described in C. I. Parl, et al, Korea-Australia Rheology Journal 2003, 15 (3), 125-130 and L. Tetley, Macromolecules 1991, 24, 117-121, which is incorporated herein in its entirety by reference. According to the present teachings, such emulsions including HIPE emulsions can be obtained and therefore benefit from emulsion volume reduction while increasing the total number of reactors (droplets). With appropriate adjustments of surfactant concentration and other mechanical parameters, the present teachings enable the preparation of such HIPE emulsions and reduce workload and processing time for subsequent down stream processes.

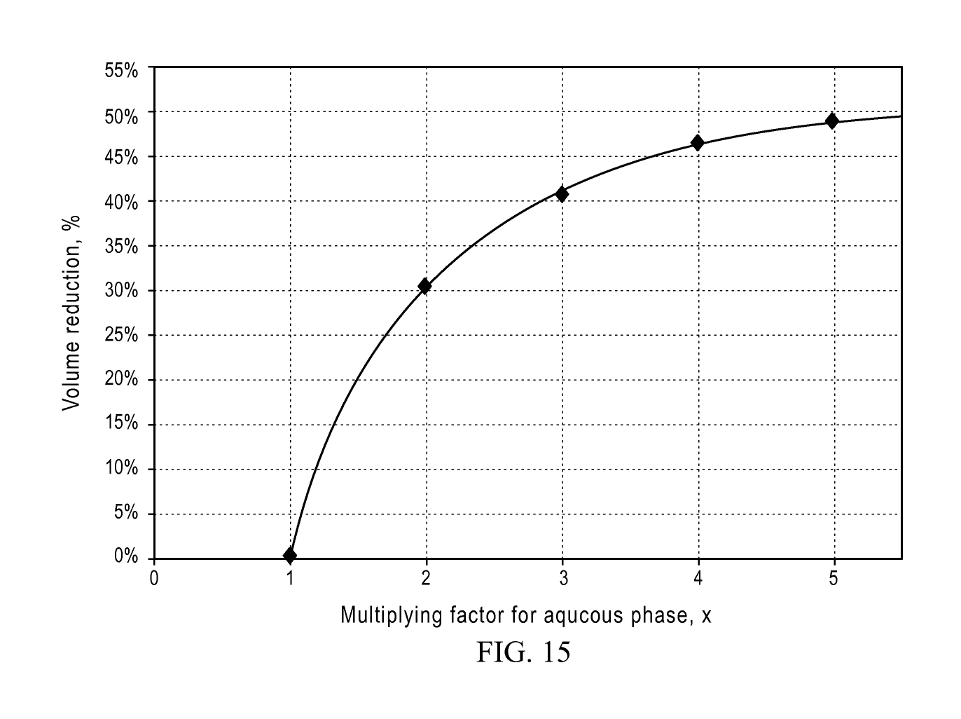

The HIPE in some embodiments can be prepared by keeping the volume of the oil phase at 9 mL as in the standard 1× emulsion while increasing its aqueous phase volume of 5_{—}6 mL by a multiplying factor of 2, 3, 4, 5, or 6. FIG. 15 shows a graph of volume reduction of HIPE emulsions. The extent of volume reduction starts to level off at 5×AQ (i.e., 5×5.6 mL aqueous phase to 1×9 mL oil phase) with a considerable increase of viscosity. FIG. 15 shows the calibration curves for 2×AQ (i.e., 2×5.6 mL aqueous phase to 1×9 mL oil phase) and 3×AQ (i.e., 3×5.6 mL aqueous phase to 1×9 mL oil phase) emulsions in approximately 20.2 mL and 25.8 mL batch sizes, respectively. A 24-mm impeller as shown in FIG. 6G was used with a D/d ratio of about 1.8. The aqueous phase comprised a KCl solution and the oil phase comprised about 9.0 mL including selected surfactants, for 2×AQ and 3×AQ emulsions. The viscosity of 4×AQ and 5×AQ emulsions is relatively high. The parameters for emulsification can be tailored accordingly.

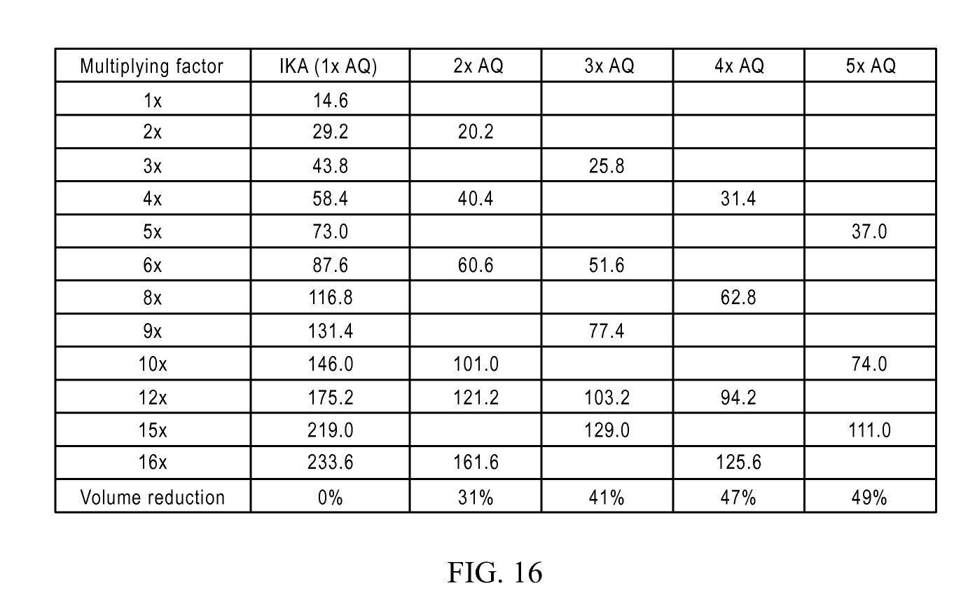

According to various embodiments of the present teachings, a basic unit of about 14.6 mL of conventional emulsion, prepared by an IKA. DT-20, comprised about 9.0 mL of mineral oil and about 5.6 mL of aqueous phase. Using this emulsion preparation as a reference, FIG. 16 depicts calculated volumes for four groups of HIPE emulsions, specifically, for 2×AQ, 3×AQ, 4×AQ, and 5×AQ emulsions. Their basic units are 20.2, 25.8, 31.4, and 37.0 mL, respectively, prepared by emulsifying about 9.0 mL of oil with about 11.2, 16.8, 22.4, and 28.0 mL of aqueous phase, respectively.

In some embodiments, referred to as a ‘plop-method’, a method of preparing an emulsion is provided that comprises contacting an aqueous phase mixture with an oil phase, in an emulsification chamber and emulsifying the combined mixture and oil phase with an impeller in the emulsification chamber to form an emulsion. The emulsification chamber is characterized by a height, an internal diameter, and a bottom, and the combined mixture and oil phase has a height (H) in the emulsification chamber. Emulsification results in the formation of a plurality of microreactors. The impeller has a diameter and the emulsification process comprises spinning the impeller at a speed of from 100 rpm to 5000 rpm, for example, from 500 rpm to 3000 rpm, from 600 rpm to 2000 rpm, from 650 rpm to 900 rpm, from 700 rpm to 850 rpm, or from 750 rpm to 800 rpm. The ratio of the internal diameter of the emulsifying chamber to the diameter of the impeller (D/d ratio) can be from 1.1:1 to 3:1. The ratio between the liquid height to the inner diameter of the emulsification chamber (H/D ratio) can be from 2:1 to 4:1, the ratio of the liquid height to the distance the impeller is arranged from the bottom of the emulsification chamber (H/h ratio) can be from 10:1 to 2:1. An impeller height h of 1.0 mm can be used for various emulsion sizes. For 116.8 mL of emulsion prepared with 72 mL of oil phase and 44.8 mL of aqueous phase in an emulsifying reactor of 80.5 mm ID, the impeller height h can be within the range of from 1.0 mm to 4.0 mm. Within the range, there are little effects on the drop size at peak, drop size distribution, and PCR performance. The microreactors can have an average drop size of from 7.0 μm to 10.0 μm, for example, from 7.5 μm to 9.5 μm, from 8.0 μm to 9.0 μm, or from 8.25 μm to 8.75 μm.

In some embodiments, the ratio D/d is from 1.5:1 to 3:1, the ratio H/h can be from 1:1 to 10:1, from 2:1 to 8:1, or from 3:1 to 6:1. The emulsifying can comprise spinning the impeller at a speed of from 700 rpm to 850 rpm, the emulsion has a volume of at least 100 mL, and the microreactors have an average drop size of from 8.0 μm to 9.0 μm.

According to various embodiments of the present teachings, a method of making a water-in-oil emulsion is provided that comprises: adding a volume of oil to a round wall container; spinning an impeller disposed in the round wall container in the volume of oil at a selected constant rpm, such that a stable vortex exists; adding a volume of aqueous solution to the stable vortex; and, after adding the volume of aqueous solution, continuing to spin the impeller in the combined volumes of oil and aqueous solution for a selected time period, thereby forming a water-in-oil emulsion in the round-wall container. The water-in-oil emulsion can be stable when thermocycled and has a selected drop diameter distribution.

According to the present teachings the emulsion preparation apparatus provides a robust and cost-effective method for emulsion generation. The apparatus is user friendly with a wide dynamic range in batch size with can be scaled up or down as desired. The apparatus also provides reproducible drop size and drop size distributions amenable to variations in aqueous-to-oil ratios to thereby allow emulsion volume reduction without a substantial loss of the total number of microreactors. Furthermore, such an apparatus is amenable to automation and may be implemented in existing workflows for emulsion preparation.

Emulsion PCR (ePCR) can be used as a sample preparation step in next generation sequencing protocols such as that performed in connection with the Applied Biosystems (Foster City, Calif.) SOLiD sequencing platform. The ePCR-based reactions for sequencing applications can use a significantly large volume reaction on the order of from about 5 mL to about 150 mL, or more.

For large volume PCR reactions, including ePCR reactions, the heat transfer characteristics for a large volume reaction may be significantly different from that of a conventional small volume reaction. Additionally, conventional PCR reactions have heat conduction properties that more closely resemble water as compared to ePCR reactions which contain a non-aqueous or oil-based component, significantly altering the heat transfer characteristics of the reaction. Consequently, predicting or modeling the fluidic behavior of an ePCR reaction using conventional predominately aqueous based information may give rise to various problems. Thermocycler temperature programming can be made to take this into account and can be optimized for the amplification of emulsion PCR samples.

In some embodiments, a large volume PCR or ePCR reaction is carried out using a minimum number of pipetting operations, to reduce the amount of labor involved as well as minimize sample loss. Consequently, such amplifications reactions in which relatively large volumes are to be processed stand to benefit from the system, methods, and components of the present teachings.

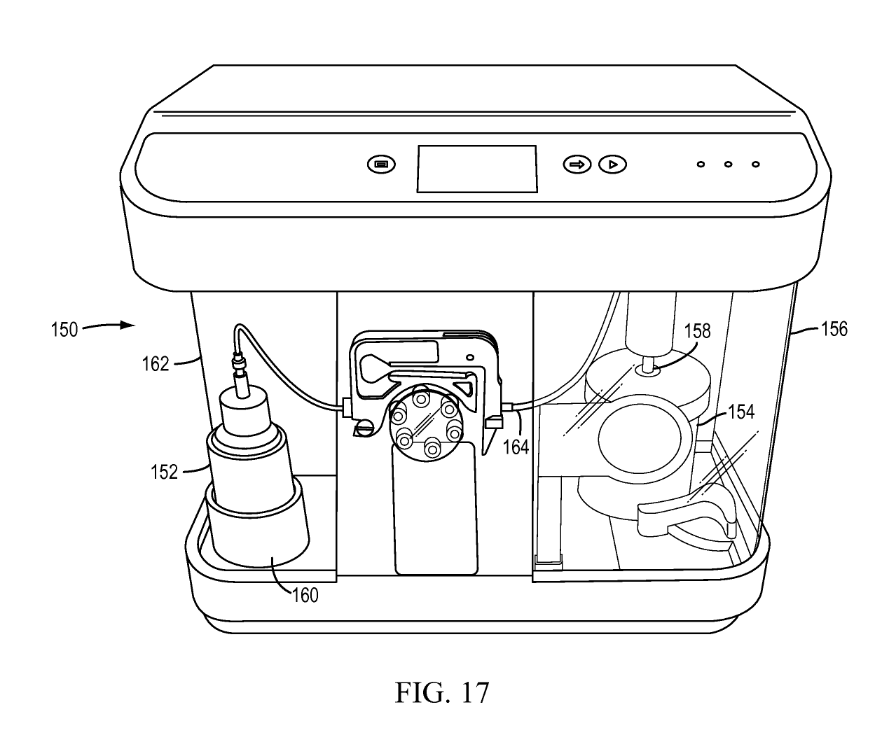

FIG. 17 shows a SOLiD EZ Bead™ emulsifier 150 according to various embodiments of the present teachings. An aqueous phase is placed in a container 152 on the left side of emulsifier 150, and an oil phase for emulsion formation is placed in a container 154 on the right side of emulsifier 150 behind a safety door 156. Once emulsifier 150 is activated, an impeller 158 spins the oil to a scale-based, pre-determined rpm and the aqueous phase is carefully metered and delivered via a peristaltic pump 160 in the center of emulsifier 150. All consumables in the system, including containers 152 and 154 and tubing 162 and 164, can be disposable to minimize cross-contamination. Containers 152 and 154 can comprise plastic, a polyalkylene material, a polypropylene material, or the like. Tubing 162 and 164 can comprise a plastic material, a silicone material, a polyalkylene material, a polypropylene material, or the like. The internal surfaces of the instrument can be designed to be easily wiped for cleaning.

In some embodiments, a sequencing system is provided that exhibits increased sequencing throughput by several orders of magnitude over gel based systems and can be instrumental in improving understanding of genomics and human disease. In some embodiments, the present teachings give end-users a most cost-effective sequencing platform.

According to various embodiments, the present teachings are capable of generating over 1×10¹° aqueous droplets in 10 mL of emulsions using 4 mL of aqueous phase and 6 mL of oil phase, in less than 15 minutes, for example, 1.21×10¹² aqueous droplets in 14.6 mL of emulsions using 5.6 mL of aqueous phase and 9 mL of oil phase, in less than 15 minutes. In an example, the emulsion batch size can be scaled up to 130 mL using 49.9 mL of aqueous phase and 80.1 mL of oil phase and a run time of 20 minutes.

In some embodiments, a system is provided that automates much of the workflow and greatly reduces the overall hands-on time regardless of scale. An exemplary system has been named EZ Bead™ to underscore the user friendliness and simplicity of operation. The EZ Bead™ system comprises three modules, the EZ Bead™ Emulsifier, the EZ Bead™ Amplifier, and the EZ Bead™ Enricher. Each module addresses key processes in the workflow: emulsion preparation, emulsion thermocycling, and bead break to templated bead enrichment.

In some embodiments, the SOLiD EZ Bead™ system is scalable; therefore, increasing the flexibility of the system to suit the throughput needs of the customer. Depending on scale chosen, the nominal bead outputs can be 125 million, 250 million and 1 billion enriched templated beads. The hands-on time for the operator can be 45 minutes regardless of scale versus a manual process for full scale which can be 145 minutes and 340 minutes for the macro scale (8 full scale preparations). The overall time for enriched templated bead preparation can be 7-8 hours depending on the input library. Overall, the EZ Bead™ system offers advantages in bead yield and bead purity compared to the manual templated bead process.

In some embodiments, the present teachings provide researchers with a cost-effective sequencing solution with unprecedented accuracy.

In some embodiments, an automated method of preparing templated beads is provided. In order to provide a user with maximum flexibility to meet their throughput needs, the system is broken into three modules. These include the SOLiD EZ Bead™ Emulsifier, SOLiD EZ Bead™ Amplifier, SOLiD EZ Bead™ Enricher. Each module can be processed in a short amount of time, for example, less than 15 minutes hands-on time. The method can use plastic consumables designed to be disposable and buffers in pre-filled racks that are easy to load. The SOLiD EZ Bead™ system provides a user with major advantages of cost reduction, time savings, and error reduction.

In some embodiments, to automate the bead preparation process, several novel methods are introduced into the SOLiD EZ Bead™ Enricher. In-line filters are used to non-magnetically concentrate beads and perform buffer exchanges. A dia-filtration process can be used in lieu of the manual glycerol cushion and centrifugation. Instead of sonication, beads can be de-aggregated using sheer flow through a syringe valve. These innovations allow for greater scalability and ease of use.

In some embodiments, the emulsion can be prepared using a SOLiD EZ Bead™ Emulsifier with a disposable plastic impeller. The continuous oil phase of the emulsion is a solution of emulsifiers in mineral oil, and the discrete aqueous phase (droplets) can comprise PCR components such as long mate pair template, primers, DNA polymerase, and SOLiD™ P1 beads. The aqueous phase can be delivered using a peristaltic pump. Both the aqueous phase and oil phase can be provided as master mixes.

The emulsion can be poured into a disposable plastic thermocycler pouch, sealed, and placed into a single volume SOLiD EZ Bead™ Amplifier. For long mate pair library templates, the emulsion mixture can be cycled for at least 40, at least 50, at least 60, or more cycles, for example, for 60 cycles. Following thermocycling, the emulsion mixture can be poured into a disposable container and placed in a SOLiD EZ Bead™ Enricher. The emulsion can be broken with 2-butanol, washed, and enriched with an automated column based enrichment method.

According to various embodiments, the method can comprise placing the pouch in a dual-sided thermocycler and thermally cycling the emulsion in the pouch. For example, the method can comprise subjecting the emulsion in the pouch to polymerase chain reaction using a thermal cycler and method as described, for example, in concurrently filed U.S. Pat. No. ______ to Liu et al., entitled “System Comprising Dual-Sided Thermal Cycler and Emulsion PCR in Pouch,” Attorney Docket No. 5010-480-02, which is incorporated herein in its entirety by reference.

According to various embodiments, the method can comprise enriching the temp lated beads using an enriching system and method as described, for example, in concurrently filed U.S. Pat. No. ______ to Karger et al., entitled “Column Enrichment of PCR Beads Comprising Tethered Amplicons,” Attorney Docket No. 5010-480-03, which is incorporated herein in its entirety by reference.

Beads can be analyzed for clonality (pre- and post P2% purity) and template loading using a calibrated BD FACS following hybridization with Cy3 labeled anti-P2 oligos. Bead quantities can be determined using a Tecan Saphire. The size distribution of emulsion reactors can be were determined using a MicroTrac particle sizer. WFA and sequencing analyses can be performed on beads manually 3′ modified and array deposited.

Taken together it will be appreciated that the disclosed systems and methods of the present teachings provide an enhanced mechanism by which to conduct PCR and ePCR reactions in relatively large volumes using easy to fabricate sample chambers with improved heat transfer characteristics.

It is to be understood that although DNA is referred to of herein, the present teachings also apply to reactions with and emulsions containing RNA, PNA, other nucleic acid molecules, other template molecules, other reactants, or combinations thereof, instead of or in addition to DNA.

It is to be understood that each of the publications referenced herein is independently incorporated herein in its entirety by reference.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “less than 10” includes any and all subranges between (and including) the minimum value of zero and the maximum value of 10, that is, any and all subranges having a minimum value of equal to or greater than zero and a maximum value of equal to or less than 10, as illustrated by the range of from 1 to 5.

It is noted that, as used in this specification and the appended claims, the singular forms “a,” “an,” and “the,” include plural referents unless expressly and unequivocally limited to one referent. As used herein, the term “include” and its grammatical variants are intended to be non-limiting, such that recitation of items in a list is not to the exclusion of other like items that can be substituted or added to the listed items.

It will be apparent to those skilled in the art that various modifications and variations can be made to the devices, systems, and methods of the present disclosure without departing from the scope its teachings. Other embodiments of the disclosure will be apparent to those skilled in the art from consideration of the specification and practice of the teachings disclosed herein. It is intended that the specification and examples be considered exemplary only.

What is claimed is:

1. A method of preparing an emulsion, comprising:
mixing together an aqueous phase solution, a plurality of template beads, a library of templates from a sample, DNA polymerase, and a pair of primers, to form a mixture;
contacting the mixture with an oil phase;
emulsifying the mixture to form an emulsion comprising a plurality of microreactors;
disposing the emulsion in a pouch; and
subjecting the emulsion in the pouch to conditions that cause templates from the library of templates to be bound to respective template beads of the plurality of template beads.

2. The method of claim 1, further comprising thermally cycling the emulsion to cause respective polymerase chain reactions in respective microreactors of the plurality of microreactors, to form a plurality of templated beads each comprising a plurality of amplicons of a respective template attached thereto.

3. The method of claim 2, further comprising breaking the emulsion to release the templated beads from the microreactors.

4. The method of claim 3, further comprising collecting the released templated beads.

5. The method of claim 4, further comprising washing the collected templated beads.

6. The method of claim 4, further comprising enriching the collected templated beads to form enriched beads.

7. The method of claim 6, further comprising eluting the enriched templated beads.

8. The method of claim 3, wherein the breaking comprises contacting the emulsion with butanol.

9. The method of claim 1, wherein the pouch comprises a plastic material and the method further comprises heat-sealing the pouch.

10. The method of claim 1, further comprising:
placing the pouch in a dual-sided thermocyler; and
thermally cycling the emulsion in the pouch.

11. The method of claim 1, wherein the disposing the emulsion in a pouch comprises transferring the emulsion from a first container to the pouch.

12. A system comprising an emulsion in a pouch, the emulsion comprising an aqueous phase solution and an oil phase, wherein
the aqueous phase solution comprises a plurality of microreactors, the plurality of microreactors collectively comprising a plurality of template beads, a library of templates from a sample, DNA polymerase, and a pair of primers, wherein at least some of the templates from the library of templates are bound to respective template beads of the plurality of template beads, and
the oil phase separates the microreactors from one another.

13. The system of claim 12, wherein at least some of the microreactors contain a templated bead that comprises a plurality of amplicons of a respective one of the templates, attached thereto.

14. The system of claim 12, wherein at least some of the microreactors are free of a templated bead that comprises a plurality of template amplicons attached thereto.

15. The system of claim 12, wherein the pouch comprises a plastic material having a thickness of 20 mils or less.

16. The system of claim 12, wherein the microreactors have an average diameter size of from 5.0 to 10.0 micrometers.

17. The system of claim 12, wherein the pouch comprises a plastic material and a heat-seal made by heat-sealing the pouch.

18. The system of claim 12, wherein the pouch comprises a top, a bottom, and an operable and closeable port at the top.

19. A method of preparing an emulsion, comprising:
mixing together an aqueous phase solution, a plurality of template beads, a library of templates from a sample, DNA polymerase, and a pair of primers, to form a mixture;
combining the mixture with an oil phase, in an emulsification chamber, the emulsification chamber having a height, an internal diameter, and a bottom, and the combined mixture and oil phase has a height (H) in the emulsification chamber; and
emulsifying the combined mixture and oil phase with an impeller in the emulsification chamber to form an emulsion comprising a plurality of microreactors, wherein the impeller has a diameter, the emulsifying comprises spinning the impeller at a speed of from 600 rpm to 2000 rpm, the ratio of the internal diameter of the emulsifying chamber to the diameter of the impeller (D/d ratio) is from about 1.1:1 to about 3:1, the ratio between the liquid height to the inner diameter of the emulsification chamber (H/D ratio) is from 2:1 to 4:1, the ratio of the liquid height to the distance the impeller is arranged from the bottom of the emulsification chamber (H/h ratio) is from 1:10 to 10:1, and the microreactors have an average drop size of from 7.0 μm to 10.0 μm.

20. The method of claim 19, wherein the ratio D/d is from 1.4:1 to 2.5:1, h is from 1.0 mm to 4.0 mm, the emulsifying comprises spinning the impeller at a speed of from 600 rpm to 850 rpm, the emulsion has a volume of at least 100 mL, and the microreactors have an average drop size of from 8.0 μm to 9.0 μm.

21. The method of claim 19, wherein the impeller comprises a disposable plastic impeller and the emulsification chamber comprises a disposable plastic container.
